(19) United States
(12) Patent Application Publication
(10) Pub. No.: US 2006/0134239 A1
(43) Pub. Date: Jun. 22, 2006

(54) Agents against microorganisms containing patchouli oil, patchouli alcohol and/or the derivatives thereof

(75) Inventors: Mirko Weide, Duesseldorf (DE); Anja Schloesser, Neuss (DE); Dirk Bockmuehl, Wuppertal (DE); Andreas Bolte, Duesseldorf (DE); Roland Breves, Mettmann (DE)

Correspondence Address: WOODCOCK WASHBURN LLP, ONE LIBERTY PLACE, 46TH FLOOR, PHILADELPHIA, PA, 19103, US

(73) Assignee: Henkel Kommanditgesellschaft Auf Aktien (Henkel KGAA), Duesseldorf (DE)

(21) Appl. No.: 11/303,630

(22) Filed: Dec. 16, 2005

Related U.S. Application Data
(63) Continuation of application No. PCT/EP04/06291, filed on Jun. 11, 2004.

(30) Foreign Application Priority Data
Jun. 17, 2003 (DE) DE 103 27 138.4
Jun. 17, 2003 (DE) DE 103 27 134.1

(51) Int. Cl. A61K 36/53; C09D 5/16

(52) U.S. Cl. 424/745; 523/122; 442/128

(57) ABSTRACT

This invention relates to preparations containing patchouli oil, patchouli alcohol and/or derivatives thereof, to the use of such preparations for inhibiting the asexual propagation of fungi and for preventing the adhesion of microorganisms to surfaces, and to filter media, adhesives, building materials, building auxiliaries, laundry detergents, cleaning compositions, rinse agents, fabric treatment compositions, hand washing compositions, manual dishwashing detergents, machine dishwashing detergents, cosmetic compositions, pharmaceutical compositions, oral hygiene compositions, dental care compositions, and denture care compositions that contain such preparations.

### CROSS REFERENCE TO RELATED APPLICATIONS

This application is a continuation of PCT/EP2004/006291, filed Jun. 11, 2004, which claims priority to DE 103 27 138.4, filed Jun. 17, 2003 and DE 103 27 134.1, filed Jun. 17, 2003, the disclosures of each of which are incorporated herein in their entireties.

The adhesion of microorganisms to surfaces is undesirable, particularly in the case of pathogenic microorganisms. Adhering microorganisms often lead to infections or re-infection in human begins, animals and plants.

Delicate textiles, such as silk or microfibers for example, are being increasingly made up into articles of clothing which can only be washed at 30 or 40° C. However, fungi such as, for example, the human-pathogenic Candida albicans are not destroyed at those temperatures. After a fungal infection in particular, these undestroyed fungi—which adhere to articles of clothing—can lead to re-infection.

In addition, denture wearers often develop oral candidosis (thrush). Fungal cells adhering to the surface of the dentures can colonize the mucous membranes, which are often predamaged by pressure points, on contact.

Hitherto, re-infection by microorganisms adhering to the clothing or to plastic surfaces has been prevented by the use of antimicrobial agents which either inhibit the growth of the microorganisms (biostatic agents) or destroy the microorganisms (biocides). The disadvantage of this is that corresponding biocides or biostatics used, for example, in laundry detergents and cleaners pollute the wastewater and hence also functionally impair the microbial stages of wastewater treatment plants. In addition, the selection pressure on the microorganisms for the buildup of resistances is greatly increased, so that, after a time, new antimicrobial agents have to be found to act against the now resistant microorganisms.

Fungi, especially molds, occur, for example, in the home where they are found in various places, for example in the kitchen or in damp rooms, for example bathrooms. Molds cause serious problems because the spores which they release into the air are often allergenic. Combating such fungi with biocides often involves an increased risk of resistance buildup so that, after a time, new antimicrobial agents have to be found to act against the now resistant microorganisms. Moreover, biocides are not always ecologically and toxicologically safe. Unwanted effects of the spread of molds include, in particular, discoloration (for example on walls, jointing compounds and other bathroom surfaces) which is caused by pigmented spores.

According to earlier, hitherto unpublished International Patent Applications PCT/EP02/14306 and PCT/EP02/14322, mono-, sesqui- and/or diterpenes and derivatives thereof can be used for inhibiting the asexual propagation of fungi and for reducing the adhesion of fungi. Farnesol is mentioned as a particularly preferred active component. The use of patchouli oil, patchouli alcohol and/or derivatives thereof for inhibiting the asexual propagation of fungi is not mentioned in those applications.

Accordingly, the problem addressed by the present invention was to overcome the disadvantages of the prior art and selectively to avoid the negative effects of sporulating fungi and to remove microorganisms from surfaces without creating increased pressure on the microorganisms to build up resistances and without polluting the surfaces or the wastewater with biocidal and/or biostatic agents or with active components in biocidal or biostatic concentrations.

This problem has been solved by preparations containing patchouli oil, patchouli alcohol and/or derivatives thereof and by their use for inhibiting the asexual propagation of fungi and for reducing the adhesion of microorganisms to surfaces.

Microorganisms in the context of the invention are understood in particular to be bacteria, fungi, viruses and algae, including bacterial endo- or exospores and spores which act as propagation structures in fungi.

Reduction of adhesion is understood to be a significant reduction in the number of adhering microorganisms cells. Ideally, adhesion is completely prevented. Preferably, the adhesion of microorganism cells is reduced or substantially completely prevented.

In the context of the invention, the term “asexual propagation” encompasses in particular sporulation, budding and fragmentation.

It has now been found that the microorganism cells do not adhere to surfaces at all, or hardly at all, in the presence of patchouli oil, patchouli alcohol and/or derivatives thereof or solutions containing these substances.

It has also surprisingly been found that the use of patchouli oil, patchouli alcohol and/or derivatives thereof on or in materials infested with fungi suppresses the asexual propagation of the fungi without destroying them.

Accordingly, the present invention relates to compositions containing patchouli oil, patchouli alcohol and/or derivatives thereof and to their use for inhibiting the asexual propagation of fungi and for reducing the adhesion of microorganisms to surfaces.

According to the invention, patchouli oil is obtained from plant parts of the patchouli bush (Pogostemon cablin or patchouli and P. heyneaus from the family of Laminaceae or Labiatae). According to the invention, the patchouli oil can be obtained by extraction with solvents or solvent mixtures, preferably organic solvents, more particularly with hydrocarbons (for example CAS 84238-39-1; CAS 90082-40-9).

Patchouli oil obtained by steam distillation from the leaves is particularly preferred (more particularly CAS 8014-09-3). Fermented leaves of the patchouli bush are preferably used for extraction. The patchouli oil is absorbed particularly well onto surfaces so that textiles in particular, but also plastic and metal surfaces, can be treated in a particularly effective and simple manner.

Besides patchouli alcohol, the patchouli oil obtained by steam distillation of the fermented leaves contains patchoulenol, patchoulenone, norpatchoulenol, nortetrapatchoulol, seychellene, α-patchoulene, β-patchoulene, α-guajene and α-bulnesene.

Any configuration isomers of patchouli alcohol may be used, although the naturally occurring (−)-patchouli alcohol is particularly preferred.

Besides esters and ethers, derivatives of patchouli alcohol are also understood to include patchoulenol, norpatchoulenol and seychellene.

Advantageously, the microorganisms are neither growth-inhibited nor destroyed by the use according to the invention; the adhesion of microorganisms to surfaces and the asexual propagation of fungi is merely inhibited or suppressed. The selection pressure to build up resistances is therefore minimal.

In addition, patchouli oil and patchouli alcohol are reputed to be toxicologically safe, so that there is no need, or at least less need, to use biocides considerably more harmful to human beings, animals and the environment. By comparison in particular with other substances which may be used, for example, for inhibiting the asexual propagation of fungi and/or for reducing the adhesion of microorganisms, such as farnesol for example, the use of patchouli oil or patchouli alcohol in the same concentrations is more advantageous from the perspective of toxicological compatibility.

Another advantage of the invention is that, compared with biocides or biostatics, these substances are effective in low final concentrations, so that there is hardly any risk of side effects.

In addition, inhibiting the asexual propagation and reducing adhesion through reduced contact of the human body with the microorganism cells, for example the respiratory tract, with molds can also lead to a reduction in the allergenic potential, particularly in living rooms.

In one particular embodiment, patchouli oil, patchouli alcohol and/or derivatives thereof are used in such final concentrations that they are not biocidal or biostatic, more particularly fungicidal or fungistatic. One particular advantage of this embodiment is that the risk of resistance to the substances used being built up is fairly minimal because the microorganisms are neither destroyed nor growth-inhibited. The concentrations at which growth is still not inhibited and the minimum inhibiting concentrations themselves may readily be determined in known manner.

In another particular embodiment, patchouli oil, patchouli alcohol and/or derivatives thereof are present in concentrations of 0.000001 to 3% by weight.

A particular advantage of this embodiment is that these active components need only be present in low concentrations to inhibit the asexual propagation of fungi, more particularly sporulation, or to reduce or substantially completely prevent the adhesion of the microorganisms to surfaces. The substances are preferably present in concentrations of 0.00001 to 1% by weight and more particularly in concentrations of 0.0001 to 0.5% by weight. Concentrations of 0.0001 to 0.1% by weight are particularly preferred. Where the substances are used for inhibiting asexual propagation, concentration ranges of 0.0001 to 0.05% by weight are particularly preferred. In the case of patchouli alcohol, concentrations of 0.0001 to 1.0% by weight are particularly preferred.

The concentrations which lead to the desired result in the end product are significantly lower than those mentioned because dilutions have to be taken into account for many products. For laundry detergents, a dilution factor (ratio of detergent concentrate to water) of 1:20 to 1:200, for example, can be expected. The dilution ratio for laundry detergents is often between 1:60 and 1:100, for example 1:80. In the final in-use solution, concentrations of 0.0001 to 1% by weight in particular have a particularly good effect (particularly for inhibiting sporulation). Concentrations of 0.001 to 0.1% by weight, for example 0.01% by weight, are preferably used.

For patchouli alcohol, for example, concentrations of 0.001 to 1.5% by weight and more especially 0.01 to 0.8% by weight would be suitable.

For the use of patchouli oil, more particularly for inhibiting the asexual propagation of fungi, concentrations of 0.001 to 1.0% by weight and more particularly 0.01 to 0.5% by weight, for example, would be suitable.

In a preferred embodiment, microorganisms are preferably understood to be bacteria and fungi. Of the fungi, the yeasts, molds and keratinophilic fungi are particularly preferred.

The active components patchouli oil, patchouli alcohol and/or derivatives thereof usable in accordance with the invention are suitable use (above all for inhibiting the asexual propagation of fungi) in particular against the fungi listed in the stock lists “DSMZ—List of Filamentous Fungi” and “DSMZ—List of Yeasts” of the DSMZ (Deutsche Stammsammlung von Mikroorganismen und Zellkulturen GmbH, Braunschweig). The lists are available on the world wide web at dsmz.de/species/fungi.htm and dsmz.de/species/yeasts.htm.

Molds in the context of the present invention are understood to be fungi which live in the soil, on human and/or animal foods or in concentrated nutrient solutions, form a typical mycelium and obtain their nutrients from organic substances which they thus decompose (saprobiontic or saprophytic organisms). In addition, they proliferate mainly asexually through spores (more particularly sporangiospores or conidiae) and form only very small, if any, sexual propagation organs.

Such fungi include, for example, species of the Ascomycota, Basidiomycota, Deuteromycota and Zygomycota classes, more particularly any species of the geni Aspergillus, Penicillium, Cladosporium and Mucor, and Stachybotrys, Phoma, Alternaria, Aureobasidium, Ulocladium, Epicoccum, Stemphyllium, Paecilomyces, Trichoderma, Scopulariopsis, Wallemia, Botrytis, Verticillium and Chaetonium

The Ascomycota include in particular all species of the geni Aspergillus, Penicillium and Cladosporium. These fungi form spores which have a strong allergenic potential on contact with the skin or the respiratory tract. The Basidiomycota include, for example, Cryptococcus neoformans. The Deuteromycota include all geni known as molds, more particularly those which cannot be assigned to the Ascomycota, Basidiomycota or Zygomycota class through the absence of a sexual stage.

Patchouli oil, patchouli alcohol and/or derivatives thereof are particularly preferred for inhibiting the asexual propagation, more particularly the sporulation, of all species of the genus Aspergillus and for reducing their adhesion to surfaces, most particularly species selected from Aspergillus aculeatus, Aspergillus albus, Aspergillus alliaceus, Aspergillus asperescens, Aspergillus awamori, Aspergillus candidus, Aspergillus carbonarius, Aspergillus carneus, Aspergillus chevalieri, Aspergillus chevalieri var. intermedius, Aspergillus clavatus, Aspergillus ficuum, Aspergillus flavipes, Aspergillus flavus, Aspergillus foetidus, Aspergillus fumigatus, Aspergillus giganteus, Aspergillus humicola, Aspergillus intermedius, Aspergillus japonicus, Aspergillus nidulans, Aspergillus niger, Aspergillus niveus, Aspergillus ochraceus, Aspergillus oryzae, Aspergillus ostianus, Aspergillus parasiticus, Aspergillus parasiticus var. globosus, Aspergillus penicillioides, Aspergillus phoenicis, Aspergillus rugulosus, Aspergillus sclerotiorum, Aspergillus sojae var. gymnosardae, Aspergillus sydowi, Aspergillus tamarii, Aspergillus terreus, Aspergillus terricola, Aspergillus toxicarius, Aspergillus unguis, Aspergillus ustus, Aspergillus versicolor, Aspergillus vitricolae and Aspergillus wentii. In a particularly preferred embodiment, the sporulation of Aspergillus flavus and Aspergillus nidulans is inhibited and their adhesion reduced or substantially completely prevented.

In a most particularly preferred embodiment, patchouli oil, patchouli alcohol and/or derivatives thereof are used for inhibiting the sporulation of species of the genus Aspergillus selected from Aspergillus flavus and Aspergillus nidulans.

In a preferred embodiment of the present invention, the patchouli oil, patchouli alcohol and/or derivatives thereof are used for inhibiting sporulation. Sporulation in the present context is understood to be the formation both of propagation forms, for example conidiae, gonitocysts, sporangiospores, arthrospores, blastospores and their associated organs (for example conidiophores), and of permanent forms (for example chlamydospores).

Since mold spores are ubiquitously present in room air, mold infestation cannot basically be prevented. However, inhibiting the sporulation of growing fungal colonies and reducing their adhesion to surfaces enables the risk of an allergy, particularly a mold allergy, to be considerably reduced and the spread of the fungus to be completely stopped or significantly delayed. Discoloration through sporulation is also greatly reduced or completely prevented.

In addition, the use of patchouli oil, patchouli alcohol and/or derivatives thereof for inhibiting sporulation has the advantage that, surprisingly, the concentration required to inhibit sporulation is considerably lower by comparison with other sesquiterpenes, for example farnesol. Thus, a comparable effect can even be achieved with a lower concentration of active component.

In addition, patchouli oil and patchouli alcohol have a woody perfume which imparts a pleasant perfume note to the corresponding substances according to the invention and may even eliminate the need to add more perfume.

Yeasts in the context of the invention are single-cell fungi which proliferate mainly by budding. Yeast fungi do not represent an independent taxonomic category in the system of fungi. Systematically, most yeasts belong to the Endomycetes. In addition, however, bud cell stages known as yeast stages occur in various other fungi in the development cycle or under certain environmental conditions. Such single-cell growth forms budding like yeasts occur among the Ascomycetes and among the Zygomycetes, Basidiomycetes and Deuteromycetes. According to the invention, all these growth forms are also understood to be yeasts.

In another particular embodiment, the adhesion of human-pathogenic fungi is reduced and/or their sexual propagation inhibited by the use of patchouli oil, patchouli alcohol and/or derivatives thereof. Such fungi include, for example, the human-pathogenic species from the Ascomycota, Basidiomycota, Deuteromycota and Zygomycota classes, more particularly the human-pathogenic forms of Candida.

The human-pathogenic Candida species colonize skin and mucous membranes, even in healthy human beings. However, in the event of vigorous propagation of the fungal cells, for example after damage to the bacterial mucous membrane flora by antibiotics, they do cause local inflammation which is also known as thrush. This inflammation occurs in the mouth and in the genital region (so-called oral or vaginal thrush). Dermal and diaper thrush are also known. The mucous membrane is reddened, lesions develop and a white coating and itching occur.

In another particularly preferred embodiment, the adhesion of fungi of the following Candida species (hereinafter abbreviated to C.), for example, is reduced: C. aaseri, C. actiscondensi, C. acutus, C. agrestis, C. albicans, C. amapae, C. anatomiae, C ancudensis, C. antarctica, C. antillancae, C. apicola, C. apis, C. aquaetextoris, C. aquatica, C. atlantica, C. atmosphaerica, C. auringiensis, C. azyma, C. beechii, C. benhamii, C. bertae, C. berthetii, C. blankii, C. boidinii, C. boleticola, C. bombi, C. bondarzewiae, C. brumptii, C. buffonii, C. buinensis, C. cacaoi, C. cantarellii, C. capsuligena, C. cariosilignicola, C. caseinolytica, C. castellii, C. catenulata, C. chalmersi, C. chilensis, C. chiropterorum, C. ciferii, C. claussenii, C. coipomensis, C. colliculosa, C. conglobata, C. curiosa, C. cylindracea, C. dendrica, C. dendronema, C. deserticola, C. diddensiae, C. diffluens, C. diversa, C. drymisii, C. dubliniensis, C. edax, C. entomophila, C. eremophila, C. ergatensis, C. ernobii, C. etchellsii, C. etchellsii, C. ethanolica, C. ethanothermophilum, C. evantina, C. fabianii, C. famata, C. fennica, C. flareri, C fluviotilis, C. fragariorum, C. fragi, C. fragicola, C. freyschussii, C. friedrichii, C. fructus, C. fusiformata, C. geochares, C. glabrata, C. glaebosa, C. graminis, C. gropengiesseri, C. guilliermondii, C. haemulonii, C. hellenica, C. heveanensis, C. holmii, C. homilentoma, C. humicola, C. humilis, C. iberica, C. incommunis, C. inconspicua, C. ingens, C. insectalens, C. insectamans, C. insectorum, C. intermedia, C. ishiwadae, C. japonica, C. javanica, C. karawaiewii, C. kefyr, C. kruisii, C. krusei, C. krusoides, C. lactiscondensi, C. lambica, C. laureliae, C. lipolytica, C llanquihuensis, C. lodderae, C. lusitaniae, C. magnoliae, C. malicola, C. maltosa, C. maris, C. maritima, C. melibiosica, C. melinii, C. membranaefaciens, C. mesenterica, C. methanosorbosa, C. milleri, C. mogii, C. molischiana, C. monosa, C. montana, C. mucilaginosa, C. multis-gemmis, C. musae, C. muscorum, C. mycoderma, C. naeodendra, C. nakasei, C. nemodendra, C. nitratophila, C. norvegensis, C novakii, C. oleophila, C. oregonensis, C. palmyrana, C. paludigena, C. parapsilosis, C. pararugosa, C. pelliculosa, C. peltata, C. periphelosum, C. petrohuensis, C. pignaliae, C. pintolopesii, C. pinus, C. placentae, C. polymorpha, C. populi, C. pseudotropicalis, C. psychrophila, C. pulcherrima, C. punica, C. quercitrusa, C. quercuum, C. railenensis, C. ralunensis, C. reukaufli, C. rhagii, C. rugopelliculosa, C. rugosa, C. saitoana, C. sake, C. salmanticensis, C. santamariae, C. santjacobensis, C. savonica, C. schatavii, C. sequanensis, C. shehatae, C. silvae, C. silvanorum, C. silvicultrix, C. solani, C. sonorensis, C. sophiae-reginae, C. sorboxylosa, C. spandovensis, C. sphaerica, C. stellata, C. stellatoidea, C. succiphila, C. sydowiorum, C. tanzawaensis, C. tenuis, C. tepae, C. terebra, C. torresii, C. tropicalis, C. tsuchiyae, C. tsukubaensis, C. utilis, C. valdiviana, C. valida, C. vanderwaltii, C. vartiovaarai, C. versatilis, C. vini, C. viswanathii, C. wickerhamii, C. xestobii, C. zeylanoides.

In another preferred embodiment, the adhesion of fungi of the species Rhodotorula spp., Cryptococcus spp., Exophilia spp. , Hormoconis spp. is reduced.

More particularly, the use according to the invention preferably reduces the adhesion of medically relevant forms of Candida, such as for example C. albicans, C. boidinii, C. catenulata, C. ciferii, C. dubliniensis, C. glabrata, C. guilliermondii, C. haemulonii, C. kefyr, C. krusei, C. lipolytica, C. lusitaniae, C. norvegensis, C. parapsilosis, C. pulcherrima, C. rugosa, C. tropicalis, C. utilis, C. viswanathii. C. albicans, C. stellatoidea, C. tropicalis, C. glabrata and C. parapsilosis are particularly preferred. The mycelium form of Candida is regarded as the human-pathogenic form of the fungus. Reducing the adhesion of Candida, for example to fabrics or plastics, reduces the risk of re-infection without increasing the build-up of resistances.

Keratinophilic fungi are understood to be skin and/or hair fungi which grow in horny skin and its appendages (especially hair and/or nails), more particularly dermatophytes and any species of the genus Malassezia. In the context of the invention, dermatophytes are understood in particular to be any species of the geni Trichophyton, Microsporum and Epidermophyton.

The keratinophilic fungus Malassezia, a yeast fungus, causes increased flaking of the skin, for example on the head (dandruff). In addition, this organism is considered to be the cause of the skin disease Pityriasis versicolor. Accordingly, it is of particular advantage to reduce or largely prevent the adhesion of Malassezia, more particularly the species M. furfur (also known as Pityrosporum ovale), M. pachydermatis, M. sympodialis and/or M. globosa.

In a preferred embodiment, the keratinophilic fungi are selected from Trichophyton mentagrophytes, T. rubrum, T. asteroides, T. concentrium, T. equinum, T. meginii, T. gallinae, T. tonsurans, T. schoenleinii, T. terrestre, T. verrucosum, T. violaceum, Microsporum canis, Microsporum audounii, M. gypseum, Epiderrnophyton flossocum, Malassezia furfur, M. sympodialis, M. globosa and M. pachydermatis.

In another preferred embodiment, the use of patchouli oil, patchouli alcohol and/or derivatives thereof reduces the adhesion of dermatophytes to surfaces. More particularly, the dermatophytes are selected from Trichophyton mentagrophytes, T. rubrum, T. asteroides, T. concentrium, T. equinum, T. meginii, T. gallinae, T. tonsurans, T. schoenleinii, T. terrestre, T. verrucosum, T. violaceum, Microsporum canis, Microsporum audounii, M. gypseum and Epidermophyton flossocum.

In a particularly preferred embodiment, the use of patchouli oil, patchouli alcohol and/or derivatives thereof reduces the adhesion of bacteria such as, for example, the following gram-begative and gram-positive bacteria, more particularly the pathogenic bacteria Propionibacterium acnes, Stapylococcus aureus, Group A Streptococci (beta-haemolyzing S.), S. pyogenes, Corynebacterium spp. (more particularly C. tenuis, C. diphtheriae, C. minutissimum), Micrococcus spp. (more particularly M. sedentarius), Bacillus anthracis, Neisseria meningitidis, N. gonorrhoeae, Pseudomonas aeruginosa, P. pseudomallei, Borrelia burgdorferi, Treponema pallidum, Mycobacterium tuberculosis, Mycobacterium spp., Escherichia coli and Streptococcus spec. (more particularly S. gordonii, S. mutans), Actinomyces spec. (more particularly A. naeslundii), Salmonella spec., Actinobacteria (more particularly Brachybacterium spec.), alpha-Proteobacteria (more particularly Agrobacterium spec.), beta-Proteobacteria (more particularly Nitrosomonas spec.), Aquabacterium spec., Hydrogenophaga, gamma-Proteobacteria (more particularly Stenotrophomonas spec.), Xanthomonas spec (campestris), Neisseria spec., Haemophilus spec. and any of the microorganisms described by “Paster et al. J. Bac. 183, 12, 2001, 3770-3783”.

In another embodiment, the use of patchouli oil, patchouli alcohol and/or derivatives thereof reduces the adhesion of human-, animal- and/or plant-pathogenic viruses and bacteriophages.

Algae are single-cell to multi-cell, variously colored, primarily photoautotrophic plants or photoautotrophic bacteria of mostly thallophytic organization, of which the gamete- and spore-forming organs are generally single-cell and may have envelopes of sterile cells. Algae are divided into green, red, blue and brown algae according to their pigment composition, green and blue algae on facades and building materials being of particular relevance. The relevant representatives of the blue algae (cyanobacteria) are those of the geni Anabaena, Anacystis, more particularly Anacystis montana, Gloeocapsa, Lyngbia, Nostoc, Oscillatoria, more particularly Oscillatoria lutea, Phormidium, Schiszothrix and Scytonema while relevant representatives of the green algae (Chlorophyta) are those of the geni Chlorella, Choricystis, Chlamydomonas, Chlorococcum, Stichcoccus, more particularly Stichcoccus bacillaris, Ulothrix and Trentepholia, more particularly Trentepholia odorata. Now, according to the invention, the adhesion of algae to surfaces, more particularly in very damp rooms and aquariums, and to surfaces exposed to weathering, for example building materials, including in particular sealants and seals, can be prevented by the use of patchouli oil, patchouli alcohol and/or derivatives thereof.

In another particularly preferred embodiment, the adhesion of microorganisms to surfaces which frequently come into contact with the human body is reduced or substantially completely prevented. The surfaces in question are, above all, abiotic, technical (or technically produced) surfaces. Accordingly, this particular embodiment does not encompass human tissue.

If these surfaces are not properly cleaned, already affected areas of the body can be re-infected or more new infections can occur through the adhesion of microorganisms.

In one most particularly preferred embodiment, the asexual propagation and/or the adhesion of microorganisms on such surfaces as fabrics, ceramics, metals and/or plastics or in or to filter media, building materials, building auxiliaries, pelts, paper, skins, leather is/are inhibited or reduced.

More particularly, the asexual propagation and/or the adhesion of microorganisms is/are inhibited or reduced on washing, sanitary fittings, floor coverings, shoes, leather, utility articles made of rubber, dentures or false teeth. Fungal infections of mucous membranes, more particularly in the mouth and the genital region, can be treated simply and successfully with antimycotics while bacterial infections can be treated with antibiotics. However, it is very important that the surfaces contaminated with the microorganism cells, for example washing, particularly lingerie or stockings in the case of fungal cells, are freed from the microorganism cells. In the case of delicate textiles, for example silk or microfibers and synthetic fabrics, this cannot be achieved by a high washing temperature without damage to the material. The use of high-bleach heavy-duty detergents is also not recommended on account of possible fabric damage.

Reducing adhesion or asexual propagation on fabrics or plastic surfaces very often prevents re-infection of the already affected areas of the body. Reducing the adhesion of microorganisms to ceramics, plastics or metals, more particularly to prostheses or false teeth, reduces the risk of infection or re-infection without polluting the skin, the mucous membranes or the wastewaters with biocidal or biostatic or virostatic substances. Catheters and other medical instruments and/or prostheses made of plastics or metal can also be freed from the adhering microorganisms by using such substances in rinses or cleaning preparations for example.

The present invention also relates to laundry detergents, cleaners, rinse agents, hand washing preparations, manual dishwashing detergents, machine dishwashing detergents, cosmetic and/or pharmaceutical preparations and preparations for treating surfaces and/or packaging, more particularly those coming into contact with foods, filter media, building materials, building auxiliaries, textiles, pelts, paper, skins or leather containing patchouli oil, patchouli alcohol and/or derivatives thereof, more particularly for inhibiting the asexual propagation of fungi and for reducing the adhesion of microorganisms to surfaces.

The present invention also relates to filter media, building materials, building auxiliaries, textiles, pelts, paper, skins or leather which contain patchouli oil, patchouli alcohol and/or derivatives thereof and/or which have been treated with a composition according to the invention.

In another particular embodiment, patchouli oil, patchouli alcohol and/or derivatives thereof are added to laundry detergents and/or cleaners or to oral hygiene or denture cleaning products. Modern textile fibers in particular, which cannot be washed with heavy-duty detergents or at high washing temperatures, cannot be completely freed from adhering microorganisms at 30 or 40° C. One advantage of using such additives in laundry detergents/cleaners is that, despite minimal wastewater pollution and a low risk of resistance build-up, articles of clothing can be freed from the adhesion of microorganisms.

False teeth, more particularly dentures, can readily be freed from the adhesion of microorganisms by using such substances in oral hygiene, dental and/or denture care products without polluting the treated surface with highly biocidal and possibly slightly toxic substances. Patchouli oil, patchouli alcohol and/or derivatives thereof are particularly suitable for oral hygiene, dental and/or denture care.

The present invention also relates to detergents and/or cleaners containing 0.000001 to 3% by weight of patchouli oil, patchouli alcohol and/or derivatives thereof. Concentrations of 0.0001 to 1.0% by weight and more especially 0.0001 to 0.5% by weight are particularly preferred. In a most particularly preferred embodiment, the detergents/cleaners contain 0.0001 to 0.05% by weight and more especially up to 0.01% by weight of patchouli oil, patchouli alcohol and/or derivatives thereof.

These detergents/cleaners may contain relatively small quantities of the substances without polluting the wastewaters. Because they are used in concentrated form and can be diluted to the correspondingly active concentrations in the wash liquor, the active components have to be used in a correspondingly relatively high concentration. The detergents/cleaners are normally diluted with water in a ratio of 1:40 to 1:200.

According to the invention, the active components may also be added to hard-surface cleaners, for example for floors, tiles, plastics and other hard surfaces in the home, more especially in damp rooms (for example bathrooms), in public sanitary facilities, in swimming baths, saunas, sports facilities or in medical or massage practices where they can advantageously prevent the unwanted discoloration of surfaces by colored spores (for example black from Aspergillus niger). Shower curtains and other bathroom textiles and plastics can also be protected against discoloration by mold(spore)s.

Inhibiting the asexual propagation of fungi on fabrics or plastic surfaces often prevents re-infection of the already affected areas of the body. Inhibiting the asexual propagation of fungi on ceramics, plastics or metals reduces the risk of infection or re-infection without polluting the skin, the mucous membranes or the wastewaters with fungicidal or fungistatic substances. Catheters and other medical instruments and/or prostheses made of plastics or metal can also be kept largely free from fungi by using patchouli oil, patchouli alcohol and/or derivatives thereof in rinses or cleaning preparations for example.

In addition, the adhesion of microorganisms which occur particularly frequently in such regions through favorable conditions is reduced or completely prevented by such cleaning preparations. This has the advantage that infection or re-infection with such microorganisms, more particularly human-pathogenic microorganisms, is made difficult or, in the best case, is completely prevented.

Besides the pathogenic microorganisms (particularly fungi and bacteria), microorganisms found on such surfaces include, in particular, Pseudomonas aeruginosa, Salmonelle spec., Actinobacteria (more particularly Brachybacterium spec.), alpha-Proteobacteria (more particularly Agrobacterium spec.), beta-Proteobacteria (more particularly Ntrosomonas spec., Aquabacterium spec., Hydrogenophaga), gamma-Proteobacteria (more particularly Stenotrophomonas spec., Xanthomonas spec (campestris)).

In the context of the invention, laundry detergents and cleaning compositions are understood in the broadest sense to be surfactant-containing preparations in solid form (particles, powders, etc.), semisolid form (pastes, etc.), liquid form (solutions, emulsions, suspensions, gels, etc.) and gas-like form (aerosols, etc.) which, to achieve an advantageous effect in use, contain one or more surfactants, normally besides other components typical of the particular application. Examples of such surfactant-containing preparations are surfactant-containing laundry detergent preparations, surfactant-containing cleaners for hard surfaces or surfactant-containing fabric conditioning preparations which may be solid or liquid or even present in a form which comprises solid and liquid components or partial amounts of the components alongside one another.

The laundry detergents and cleaners may contain typical ingredients, such as anionic, nonionic, cationic and amphoteric surfactants, inorganic and organic builders, special polymers (for example those with co-builder properties), foam inhibitors, dyes and optionally additional perfumes, bleaching agents (for example peroxo bleaching agents and chlorine bleaching agents), bleach activators, bleach stabilizers, bleach catalysts, enzymes and redeposition inhibitors without the ingredients being confined to these groups of substances. Important other ingredients of such preparations are often washing auxiliaries including, for example, optical brighteners, UV absorbers, soil repellents, i.e. polymers which counteract the resoiling of fibers. The individual groups of substances are explained in more detail in the following.

In cases where the preparations are present at least partly in the form of shaped bodies, binders and disintegration auxiliaries may also be present.

The surfactants used may be anionic, nonionic, zwitterionic and cationic surfactants.

Suitable anionic surfactants are, for example, those of the sulfonate and sulfate type. Suitable surfactants of the sulfonate type are preferably C₉₋₁₃ alkyl benzenesulfonates, olefin sulfonates, i.e. mixtures of alkene and hydroxyalkane sulfonates, and the disulfonates obtained, for example, from C₁₂₋₁₈ monoolefins with an internal or terminal double bond by sulfonation with gaseous sulfur trioxide and subsequent alkaline or acidic hydrolysis of the sulfonation products. Other suitable surfactants of the sulfonate type are the alkane sulfonates obtained from C₁₂₋₁₈ alkanes, for example by sulfochlorination or sulfoxidation and subsequent hydrolysis or neutralization. The esters of 2-sulfofatty acids (ester sulfonates), for example the 2-sulfonated methyl esters of hydrogenated coconut oil, palm kernel oil or tallow fatty acids, are also suitable.

Other suitable anionic surfactants are sulfonated fatty acid glycerol esters. Fatty acid glycerol esters in the context of the present invention are the monoesters, diesters and triesters and mixtures thereof which are obtained where production is carried out by esterification of a monoglycerol with 1 to 3 mol fatty acid or in the transesterification of triglycerides with 0.3 to 2 mol glycerol. Preferred sulfonated fatty acid glycerol esters are the sulfonation products of saturated fatty acids containing 6 to 22 carbon atoms, for example caproic acid, caprylic acid, capric acid, myristic acid, lauric acid, palmitic acid, stearic acid or behenic acid.

Preferred alk(en)yl sulfates are the alkali metal salts and, in particular, the sodium salts of the sulfuric acid semiesters of C₁₂₋₁₈ fatty alcohols, for example cocofatty alcohol, tallow fatty alcohol, lauryl, myristyl, cetyl or stearyl alcohol, or C₁₀₋₂₀ oxoalcohols and the corresponding semiesters of secondary alcohols with the same chain length. Other preferred alk(en)yl sulfates are those with the chain length mentioned which contain a synthetic, linear alkyl chain based on a petrochemical and which are similar in their degradation behavior to the corresponding compounds based on oleochemical raw materials. C₁₂₋₁₆ alkyl sulfates, C₁₂₋₁₅ alkyl sulfates and C₁₄₋₁₅ alkyl sulfates are preferred for laundry detergents and cleaners. Other suitable anionic surfactants are 2,3-alkyl sulfates which may be produced, for example, in accordance with U.S. Pat. No. 3,234,258 or U.S. Pat. No. 5,075,041 and which are commercially obtainable as products of the Shell Oil Company under the name of DAN®.

The sulfuric acid monoesters of linear or branched C₇₋₂₁ alcohols ethoxylated with 1 to 6 mol ethylene oxide, such as 2-methyl-branched C₉₋₁₁ alcohols containing on average 3.5 mol ethylene oxide (EO) or C₁₂₋₁₈ fatty alcohols containing 1 to 4 EO, are also suitable. In view of their high foaming capacity, they are only used in relatively small quantities, for example in quantities of 1 to 5% by weight, in laundry detergents and cleaners.

Other suitable anionic surfactants are the salts of alkyl sulfosuccinic acid which are also known as sulfosuccinates or as sulfosuccinic acid esters and which represent monoesters and/or diesters of sulfosuccinic acid with alcohols, preferably fatty alcohols and, more particularly, ethoxylated fatty alcohols. Preferred sulfosuccinates contain C8-18 fatty alcohol residues or mixtures thereof. Particularly preferred sulfosuccinates contain a fatty alcohol residue derived from ethoxylated fatty alcohols which, considered in isolation, represent nonionic surfactants (for a description, see below). Of these sulfosuccinates, those of which the fatty alcohol residues are derived from narrorange ethoxylated fatty alcohols are particularly preferred. Alk(en)yl succinic acid preferably containing 8 to 18 carbon atoms in the alk(en)yl chain or salts thereof may also be used.

Other suitable anionic surfactants are, in particular, soaps. Suitable soaps are saturated fatty acid soaps, such as the salts of lauric acid, myristic acid, palmitic acid, stearic acid, hydrogenated erucic acid and behenic acid, and soap mixtures derived in particular from natural fatty acids, for example coconut oil, palm kernel oil or tallow fatty acids.

The anionic surfactants, including the soaps, may be present in the form of their sodium, potassium or ammonium salts and as soluble salts of organic bases, such as mono-, di- or triethanolamine. The anionic surfactants are preferably present in the form of their sodium or potassium salts and, more preferably, in the form of their sodium salts. The surfactants may also be used in the form of their magnesium salts.

According to the invention, preferred preparations contain 5 to 50% by weight, preferably 7.5 to 40% by weight and more preferably 15 to 25% by weight of one or more anionic surfactants.

Preferred nonionic surfactants are alkoxylated, advantageously ethoxylated, more especially primary alcohols preferably containing 8 to 18 carbon atoms and, on average, 1 to 12 mol ethylene oxide (EO) per mol alcohol, in which the alcohol component may be linear or, preferably, methyl-branched in the 2-position or may contain linear and methyl-branched residues in the form of the mixtures typically present in oxoalcohol residues. However, alcohol ethoxylates containing linear residues of alcohols of native origin with 12 to 18 carbon atoms, for example coconut oil, palm oil, tallow or oleyl alcohol, and on average 2 to 8 EO per mol alcohol are particularly preferred. Preferred ethoxylated alcohols include, for example, C₁₂₋₁₄ alcohols containing 3 EO or 4 EO, C₉₋₁₁ alcohol containing 7 EO, C₁₃₋₁₅ alcohols containing 3 EO, 5 EO, 7 EO or 8 EO, C₁₂₋₁₈ alcohols containing 3 EO, 5 EO or 7 EO and mixtures thereof, such as mixtures of C₁₂₋₁₄ alcohol containing 3 EO and C₁₂₋₁₈ alcohol containing 5 EO. The degrees of ethoxylation mentioned represent statistical mean values which, for a special product, can be a whole number or a broken number. Preferred alcohol ethoxylates have a narrow homolog distribution (narrow range ethoxylates, NRE). In addition to these nonionic surfactants, fatty alcohols containing more than 12 EO may also be used, examples including tallow fatty alcohol containing 14 EO, 25 EO, 30 EO or 40 EO.

Another class of preferred nonionic surfactants which may be used either as sole nonionic surfactant or in combination with other nonionic surfactants are alkoxylated, preferably ethoxylated or ethoxylated and propoxylated, fatty acid alkyl esters preferably containing 1 to 4 carbon atoms in the alkyl chain, more especially the fatty acid methyl esters.

Another class of nonionic surfactants which may advantageously be used are the alkyl polyglycosides (APGs). Suitable alkyl polyglycosides correspond to the general formula RO(G)_{z} where R is a linear or branched, more particularly 2-methyl-branched, saturated or unsaturated aliphatic radical containing 8 to 22 and preferably 12 to 18 carbon atoms and G stands for a glycose unit containing 5 or 6 carbon atoms, preferably glucose. The degree of glycosidation z is between 1.0 and 4.0, preferably between 1.0 and 2.0 and more preferably between 1.1 and 1.4.

Linear alkyl polyglucosides, i.e. alkyl polyglycosides in which the polyglycosyl component is a glucose unit and the alkyl component is an n-alkyl group, are preferably used.

The surfactant-containing preparations according to the invention may advantageously contain alkyl polyglycosides, APG contents of more than 0.2% by weight, based on the preparation as a whole, being preferred for laundry detergent, dishwashing detergent or cleaning preparations. Particularly preferred surfactant-containing preparations contain APGs in quantities of 0.2 to 10% by weight, preferably in quantities of 0.2 to 5% by weight and more preferably in quantities of 0.5 to 3% by weight.

Nonionic surfactants of the amine oxide type, for example N-cocoalkyl-N,N-dimethylamine oxide and N-tallowalkyl-N,N-dihydroxyethylamine oxide, and the fatty acid alkanolamide type are also suitable. The quantity in which these nonionic surfactants are used is preferably no more than the quantity in which the ethoxylated fatty alcohols are used and, more preferably, no more than half that quantity.

Other suitable surfactants are polyhydroxyfatty acid amides corresponding to formula (I):

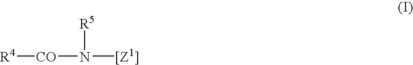

in which R⁴CO is an aliphatic acyl group containing 6 to 22 carbon atoms, R⁵ is hydrogen, an alkyl or hydroxyalkyl group containing 1 to 4 carbon atoms and [Z¹] is a linear or branched polyhydroxyalkyl group containing 3 to 10 carbon atoms and 3 to 10 hydroxyl groups. The polyhydroxyfatty acid amides are known substances which may normally be obtained by reductive amination of a reducing sugar with ammonia, an alkylamine or an alkanolamine and subsequent acylation with a fatty acid, a fatty acid alkyl ester or a fatty acid chloride.

The group of polyhydroxyfatty acid amides also includes compounds corresponding to formula (II):

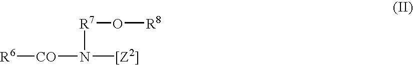

in which R⁶ is a linear or branched alkyl or alkenyl group containing 7 to 12 carbon atoms, R⁷ is a linear, branched or cyclic alkyl group or an aryl group containing 2 to 8 carbon atoms and R⁸ is a linear, branched or cyclic alkyl group or an aryl group or an oxyalkyl group containing 1 to 8 carbon atoms, C₁₋₄ alkyl or phenyl groups being preferred, and [Z²] is a linear polyhydroxyalkyl group, of which the alkyl chain is substituted by at least two hydroxyl groups, or alkoxylated, preferably ethoxylated or propoxylated, derivatives of that group.

[Z²] is preferably obtained by reductive amination of a reduced sugar, for example glucose, fructose, maltose, lactose, galactose, mannose or xylose. The N-alkoxy- or N-aryloxy-substituted compounds may then be converted into the required polyhydroxyfatty acid amides by reaction with fatty acid methyl esters in the presence of an alkoxide as catalyst, for example in accordance with the teaching of International patent application WO-A-95/07331.

In another preferred embodiment, cationic surfactants may be used in addition to anionic and nonionic surfactants.

Fabric-softening substances include, in particular, cationic surfactants. Examples of cationic surfactants are, in particular, quaternary ammonium compounds, cationic polymers and emulsifiers.

Suitable examples are quaternary ammonium compounds corresponding to formulae (III) and (IV): 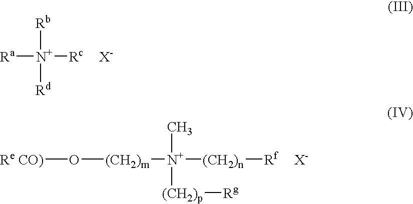
where R^{a} and R^{b} in (IV) represent an acyclic alkyl group containing 12 to 24 carbon atoms, R^{c} is a saturated C₁₋₄ alkyl or hydroxyalkyl group, R^{d} is either the same as R¹, R^{b} or R^{c} or represents an aromatic radical. X⁻ is either a halide, methosulfate, methophosphate or phosphate ion or a mixture thereof. Examples of cationic compounds corresponding to formula (III) are didecyl dimethyl ammonium chloride, ditallow dimethyl ammonium chloride or dihexadecyl ammonium chloride.

Compounds corresponding to formula (IV) are so-called esterquats. Esterquats are distinguished by excellent biodegradability. In that formula, R^{e} is an aliphatic alkyl group containing 12 to 22 carbon atoms and 0, 1, 2 or 3 double bonds, R^{f} is H, OH or O(CO)R^{h}, R^{g} independently of R^{f} stands for H, OH or O(CO)Rⁱ, R^{h} and Rⁱ independently of one another representing an aliphatic acyl group containing 12 to 22 carbon atoms and 0, 1, 2 or 3 double bonds. m, n and p independently of one another can have a value of 1, 2 or 3. X⁻ can be a halide, methosulfate, methophosphate or phosphate ion or a mixture thereof. Preferred compounds contain the group O(CO)R^{h} for R^{f} and C₁₆₋₁₈ alkyl groups for R^{c} and R^{h}. Particularly preferred compounds are those in which R^{g} is also OH. Examples of compounds corresponding to formula (IV) are methyl-N-(2-hydroxyethyl)-N,N-di(tallowacyloxyethyl)-ammonium metho-sulfate, bis-(palmitoyl)-ethyl hydroxyethyl methyl ammonium methosulfate or methyl-N,N-bis-(acyloxyethyl)-N-(2-hydroxyethyl)-ammonium methosulfate. If quaternized compounds corresponding to formula (IV) containing unsaturated alkyl chains are used, the acyl groups of which the corresponding fatty acids have an iodine value of 5 to 80, preferably 10 to 60 and more particularly 15 to 45 and which have a cis-:trans-isomer ratio (in % by weight) of greater than 30:70, preferably greater than 50:50 and more particularly greater than 70:30 are preferred. Commercially available examples are the methyl hydroxyalkyl dialkoyloxyalkyl ammonium methosulfates marketed by Stepan under the name of Stepantex® or the Cognis products known under the name of Dehyquart® or the Goldschmidt-Witco products known under the name of Rewoquat®. Other preferred compounds are the diesterquats corresponding to formula (III) which are obtainable under the name of Rewoquat® W 222 LM or CR 3099 and, besides softness, also provide for stability and color protection. 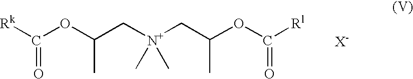
In formula (V), R^{k} and R^{l} independently of one another each represent an aliphatic acyl group containing 12 to 22 carbon atoms and 0, 1, 2 or 3 double bonds.

Besides the quaternary compounds described above, other known compounds may also be used, including for example quaternary imidazolinium compounds corresponding to formula (VI): 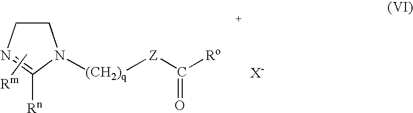
in which R^{m} represents H or a saturated alkyl group containing 1 to 4 carbon atoms, Rⁿ and R^{o} independently of one another represent an aliphatic, saturated or unsaturated alkyl group containing 12 to 18 carbon atoms, Rⁿ alternatively may also represent O(CO)R^{p}, R^{p} being an aliphatic, saturated or unsaturated alkyl group containing 12 to 18 carbon atoms, and Z is an NH group or oxygen and X⁻ is an anion. q may be an integer of 1 to 4.

Other suitable quaternary compounds correspond to formula (VII): 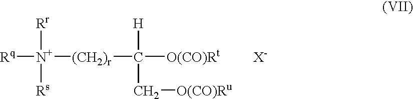
where R^{q}, R^{r} and R^{s} independently of one another represent a C₁₋₄ alkyl, alkenyl or hydroxyalkyl group, R^{t} and R^{u} independently of one another represent a C₈₋₂₈ alkyl group and r is a number of 0 to 5.

Besides the compounds corresponding to formulae (III) and (VII), short-chain, water-soluble quaternary ammonium compounds may also be used, including trihydroxyethyl methyl ammonium methosulfate or the alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride.

Protonated alkylamine compounds with a fabric-softening effect and non-quaternized protonated precursors of the cationic emulsifiers are also suitable.

Other cationic compounds suitable for use in accordance with the invention are the quaternized protein hydrolyzates.

Suitable cationic polymers are the polyquaternium polymers listed in the CTFA Cosmetic Ingredient Dictionary (The Cosmetic, Toiletry and Fragrance Association, Inc., 1997), more particularly the polyquaternium-6, polyquaternium-7 and polyquaternium-10 polymers (Ucare Polymer IR 400, Amerchol) also known as merquats, polyquaternium-4 copolymers, such as graft copolymers with a cellulose skeleton and quaternary ammonium groups attached by allyl dimethyl ammonium chloride, cationic cellulose derivatives, such as cationic guar, such as guar hydroxypropyl triammonium chloride, and similar quaternized guar derivatives (for example Cosmedia Guar, Cognis GmbH), cationic quaternary sugar derivatives (cationic alkyl polyglucosides), for example the commercial product Glucquat® (CTFA name: Lauryl Methyl Gluceth-10 Hydroxypropyl Dimonium Chloride), copolymers of PVP and dimethyl aminomethacrylate, copolymers of vinyl imidazole and vinyl pyrrolidone, aminosilicon polymers and copolymers.

Polyquaternized polymers (for example Luviquat Care, BASF) and chitin-based cationic biopolymers and derivatives thereof, for example the polymer commercially obtainable as Chitosan® (Cognis), are also suitable.

Cationic silicone oils are also suitable for the purposes of the invention, including for example the commercially available products Q2-7224 (a stabilized trimethylsilyl amodimethicone, Dow Corning), Dow Corning 929 Emulsion (containing a hydroxylamino-modified silicone which is also known as amodimethicone), SM-2059 (General Electric), SLM-55067 (Wacker), Abil®-Quat 3270 and 3272 (diquaternary polydimethylsiloxanes, quaternium-80, Goldschmidt-Rewo) and siliconequat Rewoquat® SQ 1 (Tegopren® 6922, Goldschmidt-Rewo).

Other suitable compounds correspond to formula (VIII): 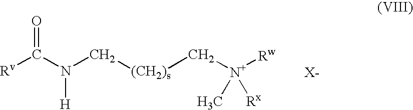
and may be alkylamidoamines in their non-quaternized form or, as illustrated, their quaternized form. In formula (VIII), R^{v} may be an aliphatic acyl group containing 12 to 22 carbon atoms and 0, 1, 2 or 3 double bonds. s may assume a value of 0 to 5. R^{w} and R^{x} independently of one another represent H, C₁₋₄ alkyl or hydroxyalkyl. Preferred compounds are fatty acid amidoamines, such as the stearylamidopropyl dimethylamine obtainable under the name of Tego Amid® S 18 or the 3-tallowamidopropyl trimethylammonium methosulfate obtainable as Stepantex® X 9124, which, besides a good conditioning effect, are also distinguished by a dye transfer inhibiting effect and by ready biodegradability.

If cationic surfactants are used, they are preferably present in the preparations in quantities of 0.01 to 10% by weight and more particularly in quantities of 0.1 to 3.0% by weight.

The total surfactant content of the compositions according to the invention may be between 5 and 50% by weight and is preferably between 10 and 35% by weight.

Next to surfactants, builders are the most important ingredients of detergents and cleaning compositions. The surfactant-containing preparations according to the invention may contain any of the builders typically used in detergents, i.e. in particular zeolites, silicates, carbonates, organic co-builders and—providing there are no ecological objections to their use—the phosphates.

Suitable crystalline layer-form sodium silicates correspond to the general formula NaMSiₓO₂ₓ₊₁. H₂O, where M is sodium or hydrogen, x is a number of 1.9 to 4 and y is a number of 0 to 20, preferred values for x being 2, 3 or 4. Crystalline layer silicates such as these are described, for example, in European patent application EP-A-0 164 514. Preferred crystalline layer silicates corresponding to the above formula are those in which M is sodium and x assumes the value 2 or 3. Both β- and δ-sodium disilicates Na₂Si₂O₅. H₂O are particularly preferred, β-sodium disilicate being obtainable, for example, by the process described in International patent application WO-A-91/08171.

Other useful builders are amorphous sodium silicates with a modulus (Na₂O:SiO₂ ratio) of 1:2 to 1:3.3, preferably 1:2 to 1:2.8 and more preferably 1:2 to 1:2.6 which dissolve with delay and exhibit multiple wash cycle properties. The delay in dissolution in relation to conventional amorphous sodium silicates can have been obtained in various ways, for example by surface treatment, compounding, compacting or by overdrying. So-called X-ray amorphous silicates, which also dissolve with delay in relation to conventional waterglasses, are described for example in German patent application DE-A-44 00 024. The products have microcrystalline regions between 10 and a few hundred nm in size, values up to at most 50 nm and more particularly up to at most 20 nm being preferred. Compacted amorphous silicates, compounded amorphous silicates and overdried X-ray-amorphous silicates are particularly preferred.

A finely crystalline, synthetic zeolite containing bound water optionally used is preferably zeolite A and/or zeolite P. Zeolite MAP® (for example Doucil A24 obtainable from Crosfield) is a particularly preferred P-type zeolite. However, zeolite X and mixtures of A, X and/or P are also suitable. According to the invention, it is also preferred to use, for example, a co-crystallizate of zeolite X and zeolite A (ca. 80% by weight zeolite X) which is marketed by CONDEA Augusta S.p.A. under the name of VEGOBOND AX® and which may be described by the following formula:
nNa₂O.(1-n)K₂O.Al₂O₃.(2-2.5)SiO₂.(3.5-5.5) H₂O.
Suitable zeolites have a mean particle size of less than 10 μm (volume distribution, as measured by the Coulter Counter Method) and contain preferably 18 to 22% by weight and more preferably 20 to 22% by weight of bound water.

The generally known phosphates may of course also be used as builders in detergents providing their use should not be avoided on ecological grounds. The sodium salts of the orthophosphates, the pyrophosphates and above all the tripolyphosphates are particularly suitable.

Suitable organic builders are, for example, polycarboxylic acids usable in the form of their sodium salts, polycarboxylic acids being understood to be carboxylic acids which carry more than one acid function, for example citric acid, adipic acid, succinic acid, glutaric acid, malic acid, tartaric acid, maleic acid, fumaric acid, sugar acids, aminocarboxylic acids, nitrilotriacetic acid (NTA), providing its use is not ecologically unsafe, and mixtures thereof. Preferred salts are the salts of the polycarboxylic acids, such as citric acid, adipic acid, succinic acid, glutaric acid, tartaric acid, sugar acids and mixtures thereof. The acids per se may also be used. Besides their builder effect, the acids also typically have the property of an acidifying component and, hence, also serve to establish a relatively low and mild pH value in surfactant-containing preparations. Citric acid, succinic acid, glutaric acid, adipic acid, gluconic acid and mixtures thereof are particularly mentioned in this regard.

Other suitable builders are polymeric polycarboxylates, for example alkali metal salts of polyacrylic acid or polymethacrylic acid, for example those with a relative molecular weight of 500 to 70,000 g/mol.

The molecular weights mentioned in this specification for polymeric polycarboxylates are weight-average molecular weights M_{w} of the particular acid form which, basically, were determined by gel permeation chromatography (GPC) using a UV detector. The measurement was made against an external polyacrylic acid standard which provides realistic molecular weight values by virtue of its structural relationship to the polymers investigated. These values differ significantly from the molecular weight values where polystyrene sulfonic acids are used as the standard. The molecular weights measured against polystyrene sulfonic acids are generally higher than the molecular weights mentioned in the present specification.

Suitable polymers are, in particular, polyacrylates which preferably have a molecular weight of 12,000 to 30,000 g/mol. Within this group, the short-chain polyacrylates which have molecular weights of 2,000 to 10,000 g/mol and more especially 3,000 to 5,000 g/mol are preferred by virtue of their superior solubility.

Other suitable polymers are copolymeric polycarboxylates, more particularly those of acrylic acid with methacrylic acid or of acrylic acid or methacrylic acid with maleic acid. Copolymers of acrylic acid with maleic acid which contain 50 to 90% by weight acrylic acid and 50 to 10% by weight maleic acid have proved to be particularly suitable. Their relative molecular weight, based on free acids, is generally in the range from 2,000 to 70,000 g/mol, preferably in the range from 20,000 to 50,000 g/mol and more particularly in the range from 30,000 to 40,000 g/mol.

The (co)polymeric polycarboxylates may be used either as powders or in the form of an aqueous solution. The content of (co)polymeric polycarboxylates in the detergents/cleaners according to the invention is preferably between 0.5 and 20% by weight and more particularly between 3 and 10% by weight.

In order to improve solubility in water, the polymers may also contain allyl sulfonic acids, such as allyloxy benzenesulfonic acid and methallyl sulfonic acid, as monomer.

Other particularly preferred polymers are biodegradable polymers of more than two different monomer units, for example those which contain salts of acrylic acid and maleic acid and vinyl alcohol or vinyl alcohol derivatives as monomers or those which contain salts of acrylic acid and 2-alkylallyl sulfonic acid and sugar derivatives as monomers.

Other preferred copolymers are those which preferably contain acrolein and acrylic acid/acrylic acid salts or acrolein and vinyl acetate as monomers.

Other preferred builders are polymeric aminodicarboxilic acids, salts or precursors thereof. Polyaspartic acids or salts and derivatives thereof, which have a bleach-stabilizing effect in addition to their co-builder properties, are particularly preferred.

Other suitable builders are polyacetals which may be obtained by reaction of dialdehydes with polyol carboxylic acids containing 5 to 7 carbon atoms and at least three hydroxy groups. Preferred polyacetals are obtained from dialdehydes, such as glyoxal, glutaraldehyde, terephthalaldehyde and mixtures thereof and from polyol carboxylic acids, such as gluconic acid and/or glucoheptonic acid.

Other suitable organic builders are dextrins, for example oligomers or polymers of carbohydrates which may be obtained by partial hydrolysis of starches. The hydrolysis may be carried out by standard methods, for example acid- or enzyme-catalyzed methods. The end products are preferably hydrolysis products with average molecular weights of 400 to 500,000 g/mol. A polysaccharide with a dextrose equivalent (DE) of 0.5 to 40 and, more particularly, 2 to 30 is preferred, the DE being an accepted measure of the reducing effect of a polysaccharide by comparison with dextrose which has a DE of 100. Both maltodextrins with a DE of 3 to 20 and dry glucose sirups with a DE of 20 to 37 and also so-called yellow dextrins and white dextrins with relatively high molecular weights of 2,000 to 30,000 may be used. A preferred dextrin is described in British patent application 94 19 091.

The oxidized derivatives of such dextrins are their reaction products with oxidizing agents which are capable of oxidizing at least one alcohol function of the saccharide ring to the carboxylic acid function. An oxidized oligosaccharide is also suitable; a product oxidized at C₆ of the saccharide ring can be particularly advantageous.

Other suitable co-builders are oxydisuccinates and other derivatives of disuccinates, preferably ethylenediamine disuccinate. Ethylenediamine-N,N′-disuccinate (EDDS) is preferably used in the form of its sodium or magnesium salts. Glycerol disuccinates and glycerol trisuccinates are also particularly preferred in this connection. The quantities used in zeolite-containing and/or silicate-containing formulations are from 3 to 15% by weight.

Other useful organic co-builders are, for example, acetylated hydroxycarboxylic acids and salts thereof which may optionally be present in lactone form and which contain at least 4 carbon atoms, at least one hydroxy group and at most two acid groups.

Another class of substances with co-builder properties are the phosphonates, more particularly hydroxyalkane and aminoalkane phosphonates. Among the hydroxyalkane phosphonates, 1-hydroxyethane-1,1-diphosphonate (HEDP) is particularly important as a co-builder. It is preferably used in the form of a sodium salt, the disodium salt showing a neutral reaction and the tetrasodium salt an alkaline ration (pH 9). Preferred aminoalkane phosphonates are ethylenediamine tetramethylene phosphonate (EDTMP), diethylenetriamine pentamethylene phosphonate (DTPMP) and higher homologs thereof. They are preferably used in the form of the neutrally reacting sodium salts, for example as the hexasodium salt of EDTMP and as the hepta- and octasodium salt of DTPMP. Within the class of phosphonates, HEDP is preferably used as builder. The aminoalkane phosphonates also show a pronounced heavy metal binding capacity. Accordingly, it can be of advantage, particularly where the surfactant-containing preparations according to the invention also contain bleaching agents, to use aminoalkane phosphonates, more especially DTPMP, or mixtures of the phosphonates mentioned.

In addition, any compounds capable of forming complexes with alkaline earth metal ions may be used as co-builders.

Among the compounds yielding H₂O₂ in water which serve as bleaching agents, sodium perborate tetrahydrate and sodium perborate monohydrate are particularly important. Other useful bleaching agents are, for example, sodium percarbonate, peroxypyrophosphates, citrate perhydrates and H₂O₂-yielding peracidic salts or peracids, such as perbenzoates, peroxophthalates, diperazelaic acid, phthaloiminoperacid or diperdodecane dioic acid. If detergent or bleaching preparations for dishwashing machines are being produced, bleaching agents from the group of organic bleaches may also be used. Typical organic bleaching agents are diacyl peroxides, such as dibenzoyl peroxide for example. Other typical organic bleaching agents are the peroxy acids, of which alkyl peroxy acids and aryl peroxy acids are particularly mentioned as examples. Preferred representatives are (a) peroxybenzoic acid and ring-substituted derivatives thereof, such as alkyl peroxybenzoic acids, but also peroxy-α-naphthoic acid and magnesium monoperphthalate, (b) aliphatic or substituted aliphatic peroxy acids, such as peroxylauric acid, peroxystearic acid, ε-phthalimidoperoxycaproic acid [phthaloiminoperoxyhexanoic acid (PAP)], o-carboxybenzamidoperoxycaproic acid, N-nonenylamidoperadipic acid and N-nonenylamidopersuccinates and (c) aliphatic and araliphatic peroxydicarboxylic acids, such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid).

In order to obtain an improved bleaching effect where washing is carried out at temperatures of 60° C. or lower, bleach activators may be incorporated in the surfactant-containing preparations. The bleach activators may be compounds which form aliphatic peroxocarboxylic acids containing preferably 1 to 10 carbon atoms and more preferably 2 to 4 carbon atoms and/or optionally substituted perbenzoic acid under perhydrolysis conditions. Substances bearing O— and/or N-acyl groups with the number of carbon atoms mentioned and/or optionally substituted benzoyl groups are suitable. Preferred bleach activators are polyacylated alkylenediamines, more particularly tetraacetyl ethylenediamine (TAED), acylated triazine derivatives, more particularly 1,5-diacetyl-2,4-dioxohexahydro-1,3,5-triazine (DADHT), acylated glycolurils, more particularly tetraacetyl glycoluril (TAGU), N-acylimides, more particularly N-nonanoyl succinimide (NOSI), acylated phenol sulfonates, more particularly n-nonanoyl or isononanoyloxybenzenesulfonate (n- or iso-NOBS), carboxylic anhydrides, more particularly phthalic anhydride, acylated polyhydric alcohols, more particularly triacetin, ethylene glycol diacetate and 2,5-diacetoxy-2,5-dihydrofuran.

In addition to or instead of the conventional bleach activators mentioned above, so-called bleach catalysts may also be incorporated in the surfactant-containing preparations. Bleach catalysts are bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and cobalt-, iron-, copper- and ruthenium-ammine complexes may also be used as bleach catalysts.

Suitable enzymes are those from the class of proteases, lipases, amylases, cellulases or mixtures thereof. Enzymes obtained from bacterial strains or fungi, such as Bacillus subtilis, Bacillus licheniformis and Streptomyces griseus, are particularly suitable. Proteases of the subtilisin type are preferred, proteases obtained from Bacillus lentus being particularly preferred. Enzyme mixtures, for example of protease and amylase or protease and lipase or protease and cellulase or of cellulase and lipase or of protease, amylase and lipase or of protease, lipase and cellulase, but especially cellulase-containing mixtures, are of particular interest. Peroxidases or oxidases have also proved to be suitable in some cases. The enzymes may be adsorbed to supports and/or encapsulated in membrane materials to protect them against premature decomposition. The percentage content of the enzymes, enzyme mixtures or enzyme granules in the surfactant-containing preparations according to the invention may be, for example, from about 0.1 to 5% by weight and is preferably from 0.1 to about 2% by weight.

A preferred group of suitable additives are optical brighteners. The optical brighteners typically used in laundry detergents may be used. Examples of optical brighteners are derivatives of diamino-stilbenedisulfonic acid or alkali metal salts thereof. Suitable optical brighteners are, for example, salts of 4,4′-bis-(2-anilino-4-morpholino-1,3,5-triazinyl-6-amino)-stilbene-2,2′-disulfonic acid or compounds of similar composition which contain a diethanolamino group, a methylamino group, an anilino group or a 2-methoxyethylamino group instead of the morpholino group. In addition, brighteners of the substituted diphenyl styryl type, for example alkali metal salts of 4,4′-bis-(2-sulfostyryl)-diphenyl, 4,4′-bis-(4-chloro-3-sulfostyryl)-diphenyl or 4-(4-chlorostyryl)-4′-(2-sulfostyryl)-diphenyl, may also be present in the part-portions (detersive preparations) of the surfactant-containing preparations according to the invention. Mixtures of the brighteners mentioned above may also be used.

Another group of additives preferred for the purposes of the invention are UV absorbers. UV absorbers can be absorbed onto the treated textiles and improve the light stability of the fibers and/or the light stability of the other formulation ingredients. UV absorbers are organic substances (light filters) which are capable of absorbing ultraviolet rays and of releasing the energy absorbed in the form of longer-wave radiation, for example heat. Compounds which possess these desired properties are, for example, the compounds which act by radiationless deactivation and derivatives of benzophenone with substituents in the 2- and/or 4-position. Other suitable UV absorbers are substituted benzotriazoles such as, for example, the water-soluble benzenesulfonic acid-3-(2H-benzotriazol-2-yl)-4-hydroxy-5-(methylpropyl)-mono-sodium salt (Cibafast® H), 3-phenyl-substituted acrylates (cinnamic acid derivatives), optionally with cyano groups in the 2-position, salicylates, organic Ni complexes and natural substances, such as umbelliferone and the body's own urocanic acid. Particular significance attaches to the biphenyl and, above all, stilbene derivatives described, for example, in EP 0728749 A which are commercially available as Tinosorb® FD and Tinosorb® FR ex Ciba. Suitable UV-B absorbers include 3-benzylidene camphor or 3-benzylidene norcamphor and derivatives thereof, for example 3-(4-methylbenzylidene)-camphor as described in EP-B1 0693471; 4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene); esters of salicylic acid, preferably salicylic acid-2-ethylhexyl ester, salicylic acid-4-isopropylbenzyl ester, salicylic acid homomenthyl ester; derivatives of benzophenone, preferably 2-hydroxy-4-methoxybenzo-phenone, 2-hydroxy-4-methoxy-4′-methylbenzophenone, 2,2′-dihydroxy-4-methoxybenzo-phenone; esters of benzalmalonic acid, preferably 4-methoxybenzmalonic acid di-2-ethylhexyl ester; triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB); propane-1,3-diones such as, for example, 1-(4-tert.butylphenyl)-3-(4′-methoxyphenyl)-propane-1,3-dione; ketotricyclo(5.2.1.0)decane derivatives as described in EP 0694521 B1. Other suitable UV-B absorbers are 2-phenylbenzimidazole-5-sulfonic acid and alkali metal, alkaline earth metal, ammonium, alkylammonium, alkanolammonium and glucamnmonium salts thereof; sulfonic acid derivatives of benzophenones, preferably 2-hydroxy-4-methoxybenzophenone-5-sulfonic acid and salts thereof; sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789), 1-phenyl-3-(4′-isopropylphenyl)-propane-1,3-dione and the enamine compounds described in DE 19712033 A1 (BASF). The UV-A and UV-B filters may of course also be used in the form of mixtures. Besides the soluble substances mentioned, insoluble light-blocking pigments, i.e. finely dispersed, preferably “nanoized” metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. Micronized zinc oxide is preferably used. Other suitable UV filters can be found in P. Finkel's review in SÖFW-Journal 122, 543 (1996).

The UV absorbers are normally used in quantities of 0.01% by weight to 5% by weight and preferably in quantities of 0.03% by weight to 1% by weight.

Another group of additives preferably used for the purposes of the invention are dyes, particularly water-soluble or water-dispersible dyes. Preferred dyes are those of the type that are typically used in laundry and dishwasher detergents, cleaners and fabric conditioners to improve their appearance. Dyes such as these, which are not difficult for the expert to choose, have high stability in storage, are not affected by the other ingredients of the surfactant-containing preparations or by light and do not have any pronounced substantivity for textile fibers so as not to color them. According to the invention, the dyes are present in the detergents and/or cleaners according to the invention in quantities of less than 0.01% by weight.

Another class of additives which may be incorporated in accordance with the invention in the detergents and/or cleaners are polymers. Suitable polymers are, on the one hand, polymers which show co-builder properties during washing or dishwashing, i.e. for example polyacrylic acids, even modified polyacrylic acids or corresponding copolymers. Another group of polymers are polyvinyl pyrrolidone and other redeposition inhibitors, such as copolymers of polyvinyl pyrrolidone, cellulose ethers and the like. Other preferred polymers are soil repellents which are described in detail in the following.

The detergents/cleaners may also contain soil repellents as further additives according to the invention. Soil repellents are polymers which are absorbed onto the fibers and have a positive effect on the removal of oil and fats from textiles by washing, thereby counteracting resoiling. This effect becomes particularly clear when a textile which has already been repeatedly washed with a detergent according to the invention containing this oil- and fat-dissolving component is soiled. Preferred oil- and fat-dissolving components include, for example, nonionic cellulose ethers, such as methyl cellulose and methyl hydroxypropyl cellulose containing 15 to 30% by weight of methoxy groups and 1 to 15% by weight of hydroxypropoxy groups, based on the nonionic cellulose ether, and the polymers of phthalic acid and/or terephthalic acid known from the prior art or derivatives thereof, more particularly polymers of ethylene terephthalates and/or polyethylene glycol terephthalates or anionically and/or nonionically modified derivatives thereof. Of these, the sulfonated derivatives of phthalic acid and terephthalic acid polymers are particularly preferred.

Particularly where they are liquids or gels, the preparations may also contain solvents. Examples of suitable solvents are monohydric or polyhydric alcohols containing 1 to 4 carbon atoms. Preferred alcohols are ethanol, propane-1,2-diol, glycerol and mixtures thereof. The solvents may be present in liquid preparations in a quantity of 2 to 12% by weight and more particularly between about 1 and 5% by weight, based on the final preparation.

The additives mentioned are added to the detergents and/or cleaners in quantities of up to at most 30% by weight and preferably in quantities of 2 to 20% by weight.

This list of detergent ingredients that may be present in the laundry/dishwashing detergents or cleaning compositions according to the invention is by no means complete and is merely intended to indicate the key ingredients typical of such compositions. In particular, organic solvents may also be present in the compositions where they are liquids or gels. These organic solvents may be mono- or polyhydric alcohols containing 1 to 4 carbon atoms. Preferred alcohols are ethanol, propane-1,2-diol, glycerol and mixtures of these alcohols. In preferred embodiments, the compositions contain 2 to 12% by weight of these alcohols.

In one particular embodiment, liquid or solid laundry detergents are particularly preferred. Light-duty laundry detergents suitable for the careful treatment of delicate textiles are also particularly preferred.

Fabric care preparations, more particularly fabric aftertreatment preparations, preferably fabric conditioners, softeners or tumble dryer sheets containing patchouli oil, patchouli alcohol and/or derivatives thereof are also particularly suitable.

Other ingredients may be used according to the intended application. Fabric softener compositions for rinse-cycle fabric softening are widely described in the prior art. These compositions normally contain a cationic quaternary ammonium compound dispersed in water as their active component. Depending on its active substance content, the final softener composition is either a dilute ready-to-use product (active substance contents below 7% by weight) or a so-called concentrate (active substance content above 7% by weight). By virtue of their lower volume and the resulting reduction in packaging and transportation costs, fabric softener concentrates have ecological advantages and have made increasing inroads on the market. Through the incorporation of cationic compounds which are poorly soluble in water, typical fabric softener compositions are dispersions that are milky white in appearance and not transparent. However, for reasons of product aesthetics, it can also be desirable to offer the consumer transparent, clear fabric softeners which are visually distinguished from the known products.

Fabric softeners according to the invention preferably contain the cationic surfactants described in detail in the foregoing as the fabric-softening active component. In a particularly preferred embodiment, the fabric softeners according to the invention contain so-called esterquats. Although there are a large number of possible compound within this class, the esterquats used in a particularly preferred embodiment of the invention are those obtained in known manner by reaction of trialkanolamines with a mixture of fatty acids and dicarboxylic acids, optionally subsequent alkoxylation of the reaction product and quaternization, as described in DE 195 39 846.

The esterquats produced in this way are eminently suitable for the production of portions according to the invention which may be used as fabric softeners. Since a large number of suitable products can be produced and used in the preparations according to the invention, depending on the choice of the trialkanolamine, the fatty acids and the dicarboxylic acids and also the quaternizing agent, a description of the esterquats preferably used in accordance with the invention based on their method of production is more precise than a general formula.

The components mentioned, which react with one another to form the esterquats preferably used, may be used in varying quantity ratios to one another. Preferred fabric softeners for the purposes of the invention are those which contain a reaction product of trialkanolamines with a mixture of fatty acids and dicarboxylic acids in a molar ratio of 1:10 to 10:1 and preferably 1:5 to 5:1, which was optionally alkoxylated and then quaternized in known manner, in quantities of 2 to 60, preferably 3 to 35 and more particularly 5 to 30% by weight. In a particularly preferred embodiment, triethanolamine is used, so that other preferred fabric softeners according to the invention contain a reaction product of triethanolamine with a mixture of fatty acids and dicarboxylic acids in a molar ratio of 1:10 to 10:1 and preferably 1:5 to 5:1, which was optionally alkoxylated and then quaternized in known manner, in quantities of 2 to 60, preferably 3 to 35 and more particularly 5 to 30% by weight.

The fatty acids used in the reaction mixture for the production of the esterquats may be any fatty acids obtained from vegetable or animal oils and fats. A fatty acid which is not solid at room temperature, i.e. is paste-like or liquid, may be used as the fatty acid in the reaction mixture.

Irrespective of their aggregate state, the fatty acids may be saturated or mono- to polyunsaturated. It is of course possible to use not only “pure” fatty acids, but also the technical fatty acid mixtures obtained in the hydrolysis of fats and oils, these mixtures being distinctly preferred from the economic point of view.

For example, individual species or mixtures of the following acids may be used in the reaction mixtures for the production of the esterquats for the clear water-based fabric softeners according to the invention: caprylic acid, pelargonic acid, capric acid, lauric acid, myristic acid, palmitic acid, stearic acid, octadecan-12-oleic acid, arachic acid, behenic acid, lignoceric acid, cerotic acid, melissic acid, 10-undecenoic acid, petroselic acid, petroselaidic acid, oleic acid, elaidic acid, ricinoleic acid, linolaidic acid, α- and β-elaeostearic acid, gadoleic acid, erucic acid, brassidic acid. It is of course also possible to use the fatty acids with an odd number of carbon atoms, for example undecanoic acid, tridecanoic acid, pentadecanoic acid, heptadecanoic acid, nonadecanoic acid, heneicosanoic acid, tricosanoic acid, pentacosanoic acid, heptacosanoic acid.

According to the invention, it is preferred to use fatty acids of formula XIII in the reaction mixture for the production of the esterquats, so that preferred fabric softeners contain a reaction product of trialkanolamines with a mixture of fatty acids corresponding to formula IX:
R¹—CO—OH (IX)
in which R¹—CO— is an aliphatic, linear or branched acyl group containing 6 to 22 carbon atoms and 0 and/or 1, 2 or 3 double bonds, and dicarboxylic acids in a molar ratio of 1:10 to 10:1 and preferably 1:5 to 5:1, which was optionally alkoxylated and then quaternized in known manner, in quantities of 2 to 60, preferably 3 to 25 and more particularly 5 to 30% by weight.

Dicarboxylic acids suitable for the production of esterquats to be used in the preparations according to the invention are, above all, saturated or mono- or polyunsaturated α,ω-dicarboxylic acids. Examples to be mentioned here are the saturated species oxalic acid, malonic acid, succinic acid, glutaric acid, adipic acid, pimelic acid, suberic acid, azelaic acid, sebacic acid, undecanoic and dodecanoic acid, brassylic acid, tetra- and pentadecanoic acid, thapi acid and hepta, octa- and nonadecanoic acid, eicosanoic and heneicosanoic acid and also phellogenic acid. Dicarboxylic acids preferably used in the reaction mixture are dicarboxylic acids corresponding to general formula XIII, so that preferred preparations according to the invention contain a reaction product of trialkanolamines with a mixture of fatty acids and dicarboxylic acids corresponding to formula X:
HO—OC—[X]—CO—OH (X)
in which X is an optionally hydroxysubstituted alkylene group containing 1 to 10 carbon atoms, in a molar ratio of 1:10 to 10:1 and preferably 1:5 to 5:1, which was optionally alkoxylated and then quaternized in known manner, in quantities of 2 to 60, preferably 3 to 35 and more particularly 5 to 30% by weight.

Of the large number of esterquats which can be produced and used in accordance with the invention, those in which the alkanolamine is triethanolamine and the dicarboxylic acid is adipic acid have proved to be particularly suitable. Accordingly, particularly preferred preparations for the purposes of the invention are those which contain a reaction product of triethanolamine with a mixture of fatty acids and adipic acid in a molar ratio of 1:5 to 5:1 and preferably 1:3 to 3:1, which was optionally quaternized in known manner, in quantities of 2 to 60, preferably 3 to 35 and more particularly 5 to 30% by weight.

The preparations according to the invention may also be provided with other additives, irrespective of whether they are formulated as laundry detergents, washing auxiliaries or fabric softeners. Examples to be mentioned in this regard are dye transfer inhibitors, “anti-gray” additives, easy-iron additives, additives releasing a particular perfume, soil release additives, resoiling inhibitors, antibacterial agents, UV absorbers, color fresheners, etc. A few examples are explained in the following:

Since sheet-form textiles, more particularly of rayon, rayon staple, cotton and blends thereof, can tend to crease because the individual fibers are sensitive to sagging, kinking, pressing and squeezing transversely of the fiber direction, the preparations according to the invention may contain synthetic anticrease agents, including for example synthetic products based on fatty acids, fatty acid esters, fatty acid amides, alkylol esters, alkylol amides or fatty alcohols, which are generally reacted with ethylene oxide, or products based on lecithin or modified phosphoric acid esters.

Wearing comfort can be increased by the additional use of antistatic agents which are additionally incorporated in the detergents according to the invention. Antistatic agents increase surface conductivity and thus provide for the improved dissipation of any charges which have built up. External antistatic agents are generally substances containing at least one hydrophilic molecule ligand and form a more or less hygroscopic film on the surfaces. These generally interfacially active antistatic agents may be divided into nitrogen-containing antistatics (amines, amides, quaternary ammonium compounds), phosphorus-containing antistatics (phosphoric acid esters) and sulfur-containing antistatics (alkyl sulfonates, alkyl sulfates). Lauryl (or stearyl) dimethyl benzyl ammonium chlorides are suitable as antistatic agents for textiles and as detergent additives and additionally develop a conditioning effect.

In order to improve the water absorption capacity and rewettability of the treated textiles and to make them easier to iron, silicone derivatives, for example, may be used in the preparations according to the invention. Silicone derivatives additionally improve the rinsing out behavior of the preparations according to the invention through their foam-inhibiting properties. Preferred silicone derivatives are, for example, polydialkyl and alkylaryl siloxanes where the alkyl groups contain 1 to 5 carbon atoms and are completely or partly fluorinated. Preferred silicones are polydimethyl siloxanes which may optionally be derivatized and, in that case, are aminofunctional or quaternized or contain Si—OH—, Si—H— and/or Si—Cl bonds. The preferred silicones have viscosities at 25° C. of 100 to 100,000 centistokes and may be used in quantities of 0.2 to 5% by weight, based on the detergent as a whole.

Finally, the preparations according to the invention may also contain U filters which are absorbed onto the treated textiles and which improve the light stability of the fibers. Compounds which have these desirable properties are, for example, the compounds acting by “radiationless” deactivation and derivatives of benzophenone with substituents in the 2 position and/or 4 position. Substituted benzotriazoles, 3-phenyl-substituted acrylates (cinnamic acid derivatives), optionally with cyano groups in the 2-position, salicylates, organic Ni complexes and natural substances, such as umbelliferone and the body's own urocanic acid.

The present invention also relates to packaging, more particularly for foods, filter media, building materials, building auxiliaries, textiles, pelts, paper, skins or leather which contain patchouli oil, patchouli alcohol and/or derivatives thereof and/or which have been treated with a preparation according to the invention.

The paper or packaging, surfaces, textiles, pelts, skins or leather are treated in known manner, for example by immersing or spraying the paper or the textiles, pelts, skins or leather in or with a suitably concentrated solution of a preparation according to the invention. For example, works of art on paper, parchment, wood and/or linen can be protected against or freed from infestation by microorganisms, more particularly molds. The filter media, building materials or building auxiliaries are treated, for example, by mechanical incorporation or application of a suitably concentrated solution of a preparation according to the invention in or to the filter media, building materials or building auxiliaries.

Patchouli oil and solutions of patchouli alcohol, preferably in organic solvents, may advantageously be applied to or incorporated particularly well in such building materials or building auxiliaries. Accordingly, the building materials or building auxiliaries may be subsequently treated or already treated building materials or building auxiliaries, for example sealing compounds, may readily be re-charged after prolonged use by application of the compositions according to the invention.

The building materials ans/or building auxiliaries treated in accordance with the invention are preferably selected from adhesives, sealing compounds, surfacing compounds and coating compositions, plastics, lacquers, paints, plaster, mortar, screed, concrete, insulating materials and primers. Particularly preferred building materials or building auxiliaries are jointing compounds (for example silicone-containing jointing compounds), wallpaper pastes, plaster, carpet adhesives, silicone adhesives, tile adhesives, dispersion paints and coating compositions for interiors and/or exteriors.

Sealing compounds and, more particularly, jointing compounds typically contain organic polymers and, in many cases, mineral or organic fillers and other additives.

Suitable polymers are, for example, the thermoplastic elastomers described in applicants' DE-A-3602526, preferably polyurethanes and acrylates. Suitable polymers are also mentioned in applicants' DE-A 3726547, DE-A 4029504 and DE-A 4009095 and in DE-A19704553 and DE-A 4233077, of which the full disclosures are included herein.

Sealing compounds and, more particularly, jointing compounds typically contain organic polymers and, in many cases, mineral or organic fillers and other additives.

Suitable polymers are, for example, the thermoplastic elastomers described in applicants' DE-A-3602526, preferably polyurethanes and acrylates. Suitable polymers are also mentioned in applicants' DE-A 3726547, DE-A 4029504 and DE-A 4009095 and in DE-A19704553 and DE-A 4233077, of which the full disclosures are included herein.

The sealing compounds (sealants or sealant mixtures) preferably contain 0.0001 to 1.5% by weight of patchouli oil, patchouli alcohol and/or derivatives thereof. Concentrations of 0.001 to 1.0% by weight, in particular up to 0.5% by weight, are particularly preferred.

According to the invention, the sealants according to the invention may be treated both in the uncured state and after curing at <60° C. In the context of the invention, sealants are materials conforming to DIN EN 26927, more particularly those which cure plastically or elastically as sealants. The sealants according to the invention may contain any of the additives typical of the corresponding sealing compounds, such as for example typical thickeners, reinforcing fillers, crosslinking catalysts, pigments, coupling agents or other volume extenders. Sealants containing patchouli oil, patchouli alcohol and/or derivatives thereof may be incorporated both in the final sealing compounds and in parts thereof or together with one or more components of the sealing compounds by dispersion in known manner, for example by using dispersing machines, kneaders, planetary mixers, etc., in the absence of moisture and oxygen.

Even the treatment of already cured, crosslinked sealant surfaces can be carried out by applying solutions or suspensions of the substance used in accordance with the invention so that the active component is transported into the sealing compound by swelling or diffusion.

Sealants usable in accordance with the invention may be based on silicones, urethanes and acrylates. Urethane-based sealants are disclosed, for example, in Ullmann's Encyclopedia of Industrial Chemistry (8^{th} Edition 2003, Chapter 4) and in U.S. Pat. No. 4,417,042.

Silicone sealants are known to the expert, for example from EP 0 118 030 A, EP 0 3161 591 A, EP 0 327 847 A, EP 0 553 143 A, DE 195 49 425 A and U.S. Pat. No. 4,417,042.

Examples of acrylate sealants are disclosed inter alia in WO 01/09249 and in U.S. Pat. No. 5,077,360.

Systems crosslinking at room temperature, as described for example in EP 0 327 847 and U.S. Pat. No. 5,077,360, are particularly preferred. These systems may be single- or multi-component systems (in multicomponent systems, the catalyst and crosslinking agent may be separately present, as disclosed, for example, in U.S. Pat. No. 4,891,400 and in U.S. Pat. No. 5,502,144) or other so-called silicone RVT two-component systems, more particularly platinum-free systems.

Particularly preferred systems are so-called one-component systems which contain all the ingredients for making a sealing compound, are stored in the absence of atmospheric moisture and/or oxygen and cure in situ by reacting with atmospheric oxygen. So-called silicone neutral systems, in which the reaction of crosslinking agents with the water or ambient air does not lead to corrosive, acidic, basic or odor-intensive decomposition products, are particularly preferred. Examples of such systems are disclosed in DE 195 49 425, in U.S. Pat. No. 4,417,042 and in EP 0 327 847.

The sealing compounds and, more particularly, jointing compounds may contain aqueous or organic solvents. Suitable organic solvents are hydrocarbons, such as cyclohexane, toluene or even xylene or petroleum ether. Other solvents are ketones, such as methylbutylketone, and chlorinated hydrocarbons.

The sealing compounds may also contain other rubber-like polymers, including relatively low molecular weight, commercial types of polyisobutylene, polyisoprene or even polybutadiene styrene. Degraded natural rubber or neoprene rubber may also be used. It is even possible to use types still liquid at room temperature which are commonly referred to as “liquid rubber”.

The sealing compounds according to the invention may be used to join materials of various different kinds to one another or to seal them. The materials in question are, primarily, concrete, glass, plaster and/or enamels, ceramic and china. However, moldings or profiles of aluminium, steel, zinc or even plastics, such as PVC or polyurethanes or acrylic resins, may also be joined or sealed. Finally, the sealing of wood or wood materials to various other materials is also mentioned.

The stability of jointing compounds is generally attributable to the addition of fine-particle solids—also known as fillers. These fillers may be divided into organic and inorganic types. Preferred inorganic fillers are, for example, silica, silicon dioxide (coated or uncoated), chalk (coated or uncoated) and/or zeolites. The zeolites may also act as drying agents. A suitable organic filler is, for example, PVC powder. The fillers generally make a key contribution to the sealing compound having the necessary inner cohesion after application so that it does not run or bulge out from vertical joints. The additives or fillers mentioned may be divided into pigments and thixotropicizing fillers—also known in short as thixotropicizing agents.

Suitable thixotropicizing agents are any of the known types, such as bentones, kaolins or even organic compounds, such as hydrogenated castor oil or derivatives thereof with polyfunctional amines or the reaction products of stearic acid or ricinoleic acid with ethylenediamine. It has proved to be particularly favorable to use silica, more particularly pyrolysis silica. Other suitable thixotropicizing agents are substantially swellable polymer powders, for example polyacrylonitrile, polyurethane, polyvinyl chloride, polyacrylates, polyvinyl alcohols, polyvinyl acetate and the corresponding copolymers. Particularly good results are obtained with fine-particle polyvinyl chloride powder. Besides the thixotropicizing agents, coupling agents, such as mercaptoalkyl silane for example, may also be used. It has proved to be useful in this regard to use a monomercaptoalkyl trialkoxysilane. Mercaptopropyl trimethoxysilane, for example, is commercially available.

The properties of a jointing compound can be further improved by adding other components to the polymer powder used as thixotropicizing agent. Such components fall into the category of plasticizers or swelling agents and swelling auxiliaries used for plastics.

Plasticizers from the class of phthalates, for example, may be used, more particularly for urethane- or acrylate-based sealing compounds. Examples of suitable compounds from this class are dioctyl phthalate, dibutyl phthalate and benzyl butyl phthalate. Other suitable classes of compounds are chloroparaffins, alkyl sulfonic acid esters, for example phenols or cresols, and fatty acid esters.

Suitable plasticizers for silicone sealing compounds are silicone oils, more particularly polydimethyl siloxanes, and hydrocarbons and/or mixtures thereof, more particularly hydrocarbons with a boiling point above 200° C. and more particularly above 230° C.

Suitable swelling auxiliaries are low molecular weight organic substances which are miscible with the polymer powder and the plasticizer. Representatives of swelling auxiliaries such as these can be found by the expert in the relevant textbooks on plastics and polymers. Preferred swelling auxiliaries for polyvinyl chloride powders are esters, ketones, aliphatic hydrocarbons, aromatic hydrocarbons and alkyl-substituted aromatic hydrocarbons.

The pigments and dyes used may be any of those already used for the applications in question, such as titanium dioxide, iron oxides and carbon black.

In order to improve stability in storage, stabilizers, such as benzoyl chloride, acetyl chloride, toluenesulfonic acid methyl ester, carbodiimides and/or polycarbodiimides, may be added to the sealing compounds, as already known. Olefins containing 8 to 20 carbon atoms have proved to be particularly effective stabilizers. Besides their stabilizing effect, these stabilizers can also act as plasticizers or swelling agents. Preferred stabilizers are olefins containing 8 to 18 carbon atoms, particularly if the double bond is in the 1,2-position. The best results are obtained when the molecular structure of these stabilizers is linear.

By using patchouli oil, patchouli alcohol and/or derivatives thereof in accordance, with the invention for reducing the adhesion of microorganisms, more particularly molds, to surfaces and the asexual propagation of fungi, more particularly molds, the problem of biocide resistance being built up is avoided. Where patchouli oil, patchouli alcohol and/or derivatives thereof are used in building materials and building auxiliaries susceptible to molds, more particularly in adhesives, coating compositions and sealing compounds and especially jointing compounds, several desirable effects are achieved through the reduction of mold adhesion to surfaces and through the inhibition of sporulation:
a) discoloration by pigmented spores is prevented,
b) the spread of the mold infestation is delayed,
c) the release of allergens is reduced.

In another preferred embodiment, the present invention relates to wallpaper adhesives containing 0.000001 to 3% by weight of patchouli oil, patchouli alcohol and/or derivatives thereof. Wallpaper pastes are prepared from aqueous solutions of hydrocolloids, such as methyl cellulose, methyl hydroxypropyl cellulose or water-soluble starch derivatives. Aqueous dispersions of film-forming high molecular weight, such as polyvinyl acetate, may also be used, particularly in conjunction with the cellulose and starch derivatives already mentioned.

The filter media used may be any of the known types providing they are suitable for use in water or air filter systems, for more particularly for air conditioning systems or room humidifiers or dehumidifiers. Filter materials of cellulose, glass fibers, PVC fibers, polyester fibers, polyamide fibers, more particularly nylon fibers, nonwovens, sintered materials and membrane filters are particularly mentioned.

The concentration of the patchouli oil, patchouli alcohol and/or derivatives thereof used to reduce the adhesion of microorganisms to surfaces in the compositions according to the invention may be varied within wide limits by the expert according to the conditions under which the preparations are used.

The preparations according to the invention are prepared to typical formulations well-known to the expert. Patchouli oil, patchouli alcohol and/or derivatives thereof are preferably added to the preparations produced in advance although, if desired, they may also be added during the production process.

The present invention also relates to a cosmetic preparation containing 0.000001 to 3% by weight patchouli oil, patchouli alcohol and/or derivatives thereof, more particularly for preventing the adhesion of microorganisms. These preparations preferably contain 0.00001 to 1.0% by weight , more preferably 0.0001 to 0.1% by weight and most preferably 0.0001 to 0.05% by weight patchouli oil, patchouli alcohol and/or derivatives thereof.

In a preferred embodiment, the preparation is an oral hygiene, dental care or denture care preparation.

Besides yeasts (more particularly those of the genus Candrola), bacteria of the genus Streptococcus (more particularly S. grondonii, S. mutans), Actinomyces (more particularly A. naeslundii), Neisseria and Haemophilus and all those described by Paster et al., J. Bac. 183 (12), 2001, pp. 3770-3783 are relevant to oral hygiene.

In the case of partial prostheses or dentures, the preparations may be formulated as denture cleaning tablets and mouth rinses or mouth washes or toothpastes.

The oral hygiene, dental care and/or denture care preparations according to the invention may be presented, for example, as mouth washes, gels, liquid tooth cleaning lotions, stiff toothpastes, denture cleaners or adhesive creams. To this end, the substances used in accordance with the invention have to be incorporated in a suitable carrier.

Suitable carriers even include, for example, powder-form preparations or aqueous alcoholic solutions which, as mouth washes, may contain 0 to 15% by weight ethanol, 1 to 1.5% by weight flavoring oils and 0.01 to 0.5% by weight sweeteners or, as mouth wash concentrates, 15 to 60% by weight ethanol, 0.05 to 5% by weight flavouring oils, 0.1 to 3% by weight sweeteners and optionally other auxiliaries and which are diluted with water before use. The concentration of the components has to be so high that, after dilution, the concentration does not fall below the lower limits mentioned in use.

However, other suitable carriers are gels and more or less flowable pastes which are expressed from flexible plastic containers or tubes and applied to the teeth with a toothbrush. Such products contain relatively large quantities of humectants and binders or consistency factors and polishing components. In addition, they may contain flavouring oils, sweeteners and water.

Suitable humectants are, for example, glycerol, sorbitol, xylitol, propylene glycols, polyethylene glycols or mixtures of these polyols, more particularly polyethylene glycols with molecular weights of 200 to 800 (or 400 to 2000). Sorbitol is preferably present as the humectant in a quantity of 25 to 40% by weight.

Suitable anti-scale components and demineralization inhibitors are condensed phosphates in the form of their alkali metal salts, preferably in the form of their sodium or potassium salts. Aqueous solutions of these phosphates show an alkaline reaction through hydrolytic effects. The pH of the oral hygiene, dental care and/or denture care preparations according to the invention is preferably adjusted to a value of 7.5 to 9 by addition of acid. Mixtures of various condensed phosphates or even hydrated salts of the condensed phosphates may also be used. However, the specific quantities of 2 to 12% by weight relate to the water-free salts. A sodium or potassium tripolyphosphate is preferably present as the condensed phosphate in a quantity of 5 to 10% by weight, based on the preparation as a whole.

An active component preferably present in a caries-inhibiting fluorine compound, preferably from the group of fluorides or monofluorophosphates, in a quantity of 0.1 to 0.5% by weight fluorine. Suitable fluorine compounds are, for example, sodium monofluorophosphate (Na₂PO₃F), potassium monofluorophosphate, sodium or potassium fluoride, tin fluoride or the fluoride of an organic amino compound.

Suitable binders and consistency factors are, for example, natural and synthetic water-soluble polymers, such as carragheen, tragacanth, guar, starch and nonionic derivatives thereof such as, for example, hydroxypropyl guar, hydroxyethyl starch, cellulose ethers such as, for example, hydroxyethyl cellulose or methyl hydroxypropyl cellulose, also agar agar, xanthan gum, pectins, water-soluble carboxyvinyl polymers (for example Carbopol® types), polyvinyl alcohol, polyvinyl pyrrolidone, relatively high molecular weight polyethylene glycols (molecular weight 10³ to 10⁶ D). Other substances suitable for controlling viscosity are layer silicates such as, for example, montmorillonite clays, colloidal thickening silicas, for example aerogel silicas or pyrogenic silicas.

Suitable polishing components are any of the known polishing materials, but preferably precipitated and gel silicas, aluminium hydroxide, aluminium silicate, aluminium oxide, aluminium oxide trihydrate, insoluble sodium metaphosphate, calcium pyrophosphate, calcium hydrogen phosphate, dicalcium phosphate, chalk, hydroxylapatite, hydrotalcites, talcum, magnesium aluminium silicate (Veegum®), calcium sulfate, magnesium carbonate, magnesium oxide, sodium aluminium silicates, for example zeolite A, or organic polymers, for example polymethacrylate. The polishing components are preferably used in relatively small quantities of, for example, 1 to 10% by weight.

The dental care and/or oral hygiene products may be improved in their organoleptic properties by addition of flavoring oils and sweeteners. Suitable flavoring oils are any of the natural and synthetic flavors typically found in oral hygiene, dental care and/or denture care preparations. Natural flavors may be used both in the form of the essential oils isolated from the drugs and in the form of the individual components isolated therefrom. The preparation should preferably contain at least one flavoring oil from the group consisting of peppermint oil, spearmint oil, aniseed oil, caraway oil, eucalyptus oil, fennel oil, cinnamon oil, geranium oil, sage oil, thyme oil, marjoram oil, basil oil, citrus oil, gaultheria oil or one or more components of these oils isolated from them or synthetically produced. The most important components of the oils mentioned are, for example, menthol, carvone, anethol, cineol, eugenol, cinnamaldehyde, geraniol, citronellol, linalool, salvia, thymol, terpinene, terpineol, methyl chavicol and methyl salicylate. Other suitable flavors are, for example, menthyl acetate, vanillin, ionone, linalyl acetate, rhodinol and piperitone. Suitable sweeteners are either natural sugars, such as sucrose, maltose, lactose and fructose, or synthetic sweeteners such as, for example, saccharin sodium salt, sodium cyclamate or aspartame.

Suitable surfactants are, in particular, alkyl and/or alkenyl (oligo)glycosides. Their production and use as surfactants are known, for example, from U.S. Pat. No. 3,839,318, U.S. Pat. No. 3,707,535, U.S. Pat. No. 3,547,828, DE-A-19 43 689, DE-A-20 36 472 and DE-A-30 01 064 and EP-A-77 167. So far as the glycoside unit is concerned, both monoglycosides (x=1), where a pentose or hexose unit is attached by a glycoside bond to a primary C₄₋₁₆ alcohol, and oligomeric glycosides with a degree of oligomerization x of up to 10 are suitable. The degree of oligomerization is a statistical mean value on which a homolog distribution typical of such technical products is based.

Particularly suitable alkyl and/or alkenyl (oligo)glycosides are alkyl and/or alkenyl (oligo)glucosides with the formula RO(C₆H₁₀O)ₓ—H, where R is a C₈₋₁₄ alkyl and/or alkenyl group and x has a mean value of 1 to 4. Alkyl oligoglucosides based on hydrogenated C_{12/14} cocoalcohol with a DP of 1 to 3 are particularly preferred. The alkyl and/or alkenyl glucoside surfactant may be used very sparingly, quantities of only 0.005 to 1% by weight being sufficient.

Besides the alkyl glucoside surfactants mentioned, other nonionic, ampholytic and cationic surfactants may also be present, including for example fatty alcohol polyglycol ether sulfates, monoglyceride sulfates, monoglyceride ether sulfates, mono- and/or dialkyl sulfosuccinates, fatty acid isethionates, fatty acid sarcosinates, fatty acid taurides, fatty acid glutamates, ether carboxylic acids, fatty acid glucamides, alkyl amidobetaines and/or protein fatty acid condensates, preferably based on wheat proteins. A nonionic solubilizer from the group of surface-active compounds may be necessary, in particular for solubilizing the generally water-insoluble flavoring oils. Particularly suitable nonionic solubilizers are, for example, ethoxylated fatty acid glycerides, ethoxylated fatty acid sorbitan partial esters or fatty acid partial esters of glycerol or sorbitan ethoxylates. Solubilizers from the group of ethoxylated fatty acid glycerides include, above all, products of the addition of 20 to 60 mol ethylene oxide onto mono- and diglycerides of linear C₁₂₋₁₈ fatty acids or onto triglycerides of hydroxyfatty acids, such as hydroxystearic acid or ricinoleic acid. Other suitable solubilizers are ethoxylated fatty acid sorbitan partial esters, i.e. preferably products of the addition of 20 to 60 mol ethylene oxide onto sorbitan monoesters and sorbitan diesters of C₁₂₋₁₈ fatty acids. Other suitable solubilizers are fatty acid partial esters of glycerol or sorbitan ethoxylates, i.e. preferably mono- and diesters of C₁₂₋₁₈ fatty acids and products of the addition of 20 to 60 mol ethylene oxide onto 1 mol glycerol or onto 1 mol sorbitol.

The oral hygiene, dental care and/or denture care preparations according to the invention preferably contain products of the addition of 20 to 60 mol ethylene oxide onto hydrogenated or non-hydrogenated castor oil (i.e. onto hydroxystearic acid or ricinoleic acid triglyceride), onto glycerol mono- and/or distearate or onto sorbitan mono- and/or distearate as solubilizers for any flavoring oils which may be present.

Other typical additives for oral hygiene, dental care and/or denture care are, for example,

pigments, for example titanium dioxide, and/or dyes

pH adjusters and buffers such as, for example, sodium bicarbonate, sodium citrate, sodium benzoate, citric acid, phosphoric acid or acidic salts, for example NaH₂PO₄

wound-healing and inflammation-inhibiting components such as, for example, allantoin, urea, panthenol, azulene or camomile extract

other anti-scale components such as, for example, organophosphates, for example hydroxyethane diphosphonates or azacycloheptane diphosphonate

preservatives such as, for example, sorbic acid salts, p-hydroxybenzoic acid esters

plaque inhibitors such as, for example, hexachlorophene, chlorhexidine, hexetidine, triclosan, bromchlorophene, phenyl salicyate.

In one particular embodiment, the composition is a mouth rinse, a mouth wash, a denture cleaner or a denture adhesive.

These compositions are used either without dilution or as a concentrate. Accordingly, besides the usual constituents, the concentrates and the denture cleaners preferably contain 0.001 to 1, more preferably 0.001 to 0.5 and most preferably 0.005 to 0.1% by weight of patchouli oil, patchouli alcohol and/or derivatives thereof while the mouth rinses and denture adhesives to be used without dilution preferably contain 0.0001 to 0.5, more preferably 0.0001 to 0.1 and most preferably 0.0001 to 0.05% by weight of patchouli oil, patchouli alcohol and/or derivatives thereof.

Besides the ingredients already mentioned for oral hygiene, dental care and/or denture care, per compounds such as, for example, peroxo borate, peroxo monosulfate or percarbonate are also suitable for preferred denture cleaners according to the invention, more particularly denture cleaning tablets and powders. They have the advantage that, besides a bleaching effect, they also have a deodorizing and/or disinfecting effect. The per compounds are used in denture cleaners in quantities of 0.01 to 10% by weight and more particularly in quantities of 0.5 to 5% by weight.

Suitable other ingredients are also enzymes such as, for example, proteases and carbohydrase for degrading proteins and carbohydrates. The pH may be in the range from pH 4 to 12 and is preferably in the range from pH 5 to 11.

The denture cleaning tablets require the presence of other auxiliaries, such as for example effervescing substances, for example CO₂-releasing substances, such as sodium hydrogen carbonate, fillers, for example sodium sulfate or dextrose, lubricants, for example magnesium stearate, flow regulators, such as colloidal silicon dioxide for example, and granulating aids, such as the above-mentioned high molecular weight polyethylene glycols or polyvinyl pyrrolidone.

Denture adhesives may be formulated as powders, creams, films or liquids and support the adhesion of the dentures. Suitable active components are natural and synthetic swelling substances. Besides alginates, natural swelling substances include vegetable gums such as, for example, gum arabic, tragacanth and karaya gum and also natural rubber. Alginates and synthetic swelling substances, such as for example sodium carboxymethyl cellulose, high molecular weight ethylene oxide copolymers, salts of poly(vinyl ether-co-maleic acid) and polyacrylamides, have proved to be particularly effective. Particularly suitable auxiliaries for paste-form and liquid products are hydrophobic bases, more particularly hydrocarbons, such as for example White Vaseline (DAB) or paraffin oil.

The present invention also relates to cosmetic preparations, more particularly body care preparations, containing patchouli oil, patchouli alcohol and/or derivatives thereof for reducing the adhesion of microorganisms, more particularly fungi and especially keratinophilic fungi, such as for example hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, superfatting agents, pearlizing waxes, consistency factors, thickeners, polymers, silicone compounds, fats, waxes, stabilizers, biogenic agents, deodorizers, antiperspirants, antidandruff agents, film formers, swelling agents, UV protection factors, antioxidants, hydrotropes, preservatives, insect repellents, self-tanning agents, solubilizers, perfume oils, dyes and the like as further auxiliaries and additives.

In a preferred embodiment, the cosmetic preparation is selected from body care preparations, more particularly creams, lotions, gels (more particularly for the hands and/or feet), shower, foam and/or foot baths, and hair treatment preparations, more particularly hair shampoos, hair lotions and hair care preparations.

By using patchouli oil, patchouli alcohol and/or derivatives thereof in cosmetic preparations, the adhesion of microorganisms, preferably fungi and especially keratinophilic fungi, to skin and especially human skin can advantageously be reduced.

The development of an infection of the skin or horny skin and the skin appendages can be prevented particularly effectively by the use of the cosmetic preparations according to the invention. In particular, the adhesion and hence the proliferation of, in particular, keratinophilic fungi in the skin and the nails can be prevented by lotions and skin creams, more particularly for the hands and nails, without antimicrobial substances having to be additionally used. Secondary infections of the already damaged areas of skin, more particularly by bacteria, can also be prevented in this way.

In the case of dandruff in particular, their use in hair treatment preparations, preferably hair shampoos or hair lotions, and hair care products, such as hair medications, preferably against dandruff, is particularly suitable for separating and more easily removing the causes of the dandruff from the scalp and the hair.

Typical examples of suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, x-olefin sulfonates, ether carboxylic acids, alkyl oligoglucosides, fatty acid glucamides, alkylamidobetaines and/or protein fatty acid condensates, preferably based on wheat proteins.

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

Suitable emulsifiers are, for example, nonionic surfactants from at least one of the following groups:

(1) products of the addition of 2 to 30 mol ethylene oxide and/or 0 to 5 mol propylene oxide onto linear fatty alcohols containing 8 to 22 carbon atoms, onto fatty acids containing 12 to 22 carbon atoms, onto alkylphenols containing 8 to 15 carbon atoms in the alkyl group and alkyl amines containing 8 to 22 carbon atoms in the alkyl group;

(2) C_{12/18} fatty acid monoesters and diesters of products of the addition of 1 to 30 mol ethylene oxide onto glycerol;

(3) glycerol monoesters and diesters and sorbitan monoesters and diesters of saturated and unsaturated fatty acids containing 6 to 22 carbon atoms and ethylene oxide adducts thereof;

(4) alkyl and/or alkenyl mono- and oligoglycosides containing 8 to 22 carbon atoms in the alkyl group and ethoxylated analogs thereof;

(5) products of the addition of 15 to 60 mol ethylene oxide onto castor oil and/or hydrogenated castor oil;

(6) polyol esters and, in particular, polyglycerol esters;

(7) products of the addition of 2 to 15 mol ethylene oxide onto castor oil and/or hydrogenated castor oil;

(8) partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);

(9) mono-, di- and trialkyl phosphates and mono-, di- and/or tri-PEG-alkyl phosphates and salts thereof;

(10) wool wax alcohols;

(11) polysiloxane/polyalkyl polyether copolymers and corresponding derivatives;

(12) mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol,

(13) polyalkylene glycols and

(14) glycerol carbonate.

The addition products of ethylene oxide and/or propylene oxide onto fatty alcohols, fatty acids, alkylphenols, glycerol monoesters and diesters and sorbitan monoesters and diesters of fatty acids or onto castor oil are known, commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic preparations from DE-PS 20 24 051.

Alkyl and/or alkenyl mono- and oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane(® NL), Polyglyceryl-3 Distearate (Cremophor® GS 32) and Polyglyceryl Polyricinoleate (Admul® WOL 1403), Polyglyceryl Dimerate Isostearate and mixtures thereof.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkyl-aminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Besides ampholytic emulsifiers, quaternary emulsifiers are also suitable, those of the esterquat type, preferably methyl-quaternized difatty acid triethanolamine ester salts, being particularly preferred.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxy-substituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil types (hydrophilic silicas), polysaccharides, more especially xanthan gum, guar guar, agar agar, alginates and tyloses, carboxymethyl cellulose and hydroxyethyl cellulose, also relatively high molecular weight polyethylene glycol monoesters and diesters of fatty acids, polyacrylates (for example Carbopols® from Goodrich or Synthalens® from Sigma), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-I of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Typical examples of fats are glycerides. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Metal salts of fatty acids such as, for example, magnesium, aluminium and/or zinc stearate or ricinoleate may be used as stabilizers.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors—which may optionally be added to the cosmetic preparations according to the invention in addition to the patchouli oil, patchouli alcohol and/or derivatives thereof used to inhibit the adhesion of microorganisms—are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chloro-phenoxy)-propane-1,2-diol, 3-iodo-2-propinyl butyl carbamate, chlorhexidine, 3,4,4′-trichlorocarbanilide (TTC), antibacterial perfumes, menthol, mint oil, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Enzyme inhibitors may also be added to the cosmetic preparations according to the invention. For example, esterase inhibitors are possibly suitable enzyme inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Düsseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type.

Antiperspirants reduce perspiration and thus counteract underarm wetness and body odor by influencing the activity of the eccrine sweat glands. Aqueous or water-free antiperspirant formulations typically contain the following ingredients:
(a) astringent active principles,
(b) oil components,
(c) nonionic emulsifiers,
(d) co-emulsifiers,
(e) consistency factors,
(f) auxiliaries in the form of, for example, thickeners or complexing agents and/or
(g) non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Climbazole, octopirox and zinc pyrithione may be used as antidandruff agents. The preparations according to the invention may preferably be used in combination with at least one of these antidandruff agents for controlling dandruff.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor as described in EP 0693471 B1;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.O)decane derivatives as described in EP 0694521 B1.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SOFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”). Suitable insect repellents are N,N-diethyl-m-toluamide, pentane-1,2-diol or Ethyl Butylacetylaminopropionate. A suitable self-tanning agent is dihydroxyacetone.

Suitable perfume oils are mixtures of natural and synthetic perfumes. Natural perfumes include the extracts of blossoms (lily, lavender, rose, jasmine, neroli, ylang-ylang), stems and leaves (geranium, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type.

Suitable dyes are any of the substances suitable and approved for cosmetic purposes as listed, for example, in the publication “Kosmetische Färbemittel” of the Farbstoff-kommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular formulation. The formulations may be produced by standard hot or cold processes and are preferably produced by the phase inversion temperature method.

The present invention also relates to pharmaceutical preparations for the treatment and/or prophylaxis of infections by microorganisms, preferably by bacteria and/or fungi, more preferably by fungi and most preferably by keratinophilic fungi, characterized in that they contain patchouli oil, patchouli alcohol and/or derivatives thereof

More particularly, these preparations may be topically applied to the skin and its appendages, more particularly the hair hair and the nails.

Infections by fungi, for example by Candida, and in particular by keratinophilic fungi, such as for example Trichophyton, Microsporum and Epidermophyton (foot and nail fungus) or Malassesia (Pityriasis versicolor), can thus be treated by the use of non-antimycotic substances. The preparations according to the invention are also suitable for the prophylaxis of such infections by preventing the fungi and especially the keratinophilic fungi from settling in the skin (more particularly the horny skin of the hands and feet) or in the skin appendages and freely proliferating there. The build-up of resistances of these fungi to the usual antimycotics is thus also avoided.

Areas of skin particularly vulnerable to infection are the scalp, nails, hands and feet, skin folds and in particular the clefts between toes and fingers. These infections are also known correspondingly as Tinea capitis, Tinea unguium, Tinea manuum, Tinea pedis and Tinea inguinalis.

Infections of the skin and mucous membrane, more particularly in the mouth and in the genital region, by species of the genus Candida, more particularly C. albicans, C. glabrata, C. tropicalis, C. dublininesis, can also be treated or prevented by the pharmaceutical preparations according to the invention.

The preparations according to the invention are particularly suitable for the treatment and/or prophylaxis of dermatomycoses caused by keratinophilic fungi, more particularly the dermatophytes, and candidosis caused by Candida, more particularly Candida albicans.

The pharmaceutical preparations preferably contain a physiologically compatible carrier. This comprises one or more adjuvants of the type typically used in such preparations, such as for example thickeners, moistening and/or moisturising substances, surfactants, emulsifiers, plasticizers, foam inhibitors, fats, oils, waxes, silicones, sequestering agents, anionic, cationic, nonionic or amphoteric polymers, alkalizing or acidifying agents, alcohols, polyols, softeners, adsorbents, UV filters, electrolytes, organic solvents, preservatives, bactericides, antioxidants, perfumes, flavors, dyes and pigments.

For the preferred topical application, the pharmaceutical preparations may be made up in different forms, for example creams or ointments, more particularly in water-free form, for example an oil or a balm, or even in the form of an oil-in-water or water-in-oil emulsion, which may be a cream or a milk for example, in the form of suspensions, solutions, powders or plasters. If the preparations are water-free, the carrier may be a vegetable or animal oil, a mineral oil or even a synthetic oil or mixtures of such oils. Patchouli oil and patchouli alcohol show particularly good solubility in oils and, hence, are also particularly suitable for incorporation in water-free products.

In a particularly preferred embodiment, the adhesion of keratinophilic fungi to biotic surfacess, for example skin and/or its appendages, is reduced. By this is meant, above all, skin and its apendages, such as the hair and/or nails of human beings and/or animals.

Preferably, the adhesion of fungi, particularly keratinophilic fungi, to human biotic surfaces, more particularly the human skin, is reduced by the use of patchouli oil, patchouli alcohol and/or derivatives thereof in accordance with the invention.

The adhesion of microorganisms, particularly fungi, to animals and the risk of these fungi being transferred to humans, which could be accompanied by serious inflammatory reactions, can also be reduced from the outset by the use of patchouli oil, patchouli alcohol and/or derivatives thereof in accordance with the invention. The danger of infection can thus be distinctly reduced.

### EXAMPLES

### Example 1

Effect of Patchouli Oil on the Sporulation of Aspergillus niger

Contamination of the surface of wort agar plates with 100 μl of a germ suspension (10³ CFU/ml) of Aspergillus niger (DSM 1988). Various quantities of active component (dissolved in ethanol, for final concentrations see Table 1) were added to the agar plates beforehand. The plates were incubated for 3 days at 25° C. Sporulation was visually evaluated and the sporulation rate in [%] was determined. None of the active component concentrations used inhibited the growth of the test strain. Sporulation was inhibited with increasing concentrations and was completely suppressed at 150 μm.

TABLE 1

| | Concentration of patchouoli oil [μm] | | | | | | | | |
|---|---|---|---|---|---|---|---|---|---|
| | 0 | 3 | 30 | 45 | 60 | 75 | 150 | 225 | 450 |
| Sporulation [%] | 100 | 95 | 95 | 95 | 75 | 70 | 0 | 0 | 0 |

TABLE 2
Control

| | Concentration of farnesol [μm] | | | | | |
|---|---|---|---|---|---|---|
| | 0 | 25 | 62.5 | 125 | 250 | 500 |
| Sporulation [%] | 100 | 90 | 75 | 50 | 10 | 0 |

Active component: patchouli oil obtained by steam distillation (mixture of patchouli oil from Kaders, Sensient, Polarome and Nitsche)

TABLE 3
Composition of patchouli oil
Standard analysis of patchouli oil

| % | Name | CAS-No. 1 |
|---|---|---|
| 32.0 | Patchouli alcohol | 5986-550 |
| 17.1 | Bulnesene | 3691-11-0 |
| 14.3 | Guaiene, alpha- | 654-48-6 |
| 8.2 | Seychellene | 20085-932 |
| 5.1 | Patchoulene, alpha- | 560-32-7 |
| 3.4 | Caryophyllene | 87-44-5 |
| 2.4 | Pogostol | 21698-419 |
| 2.1 | Patchoulene, beta- | 514-51-2 |
| 1.0 | Humulene, alpha- | 6753-98-6 |
| 0.3 | Caryophyllene oxide | 1139-30-6 |
| 0.3 | Copaene | 3856-25-5 |
| 0.2 | Pinene, beta- | 127-91-3 |
| 0.2 | d-Limonene | 5989-27-5 |
| 0.1 | Pinene, alpha- | 80-56-8 |
| 0.1 | Pentadecane | 629-62-9 |
| 13.2 | Unidentified components | — |

### Example 2

Effect of Patchouli Alcohol on the Sporulation of Aspergillus niger

Contamination of the surface of wort agar plates with 100 μl of a germ suspension (10³ CFU/ml) of Aspergillus niger (DSM 1988). Various quantities of active component (solutions in ethanol, for final concentrations see Table 2) were added to the agar plates beforehand. The plates were incubated for 5 days at 25° C. Sporulation was visually evaluated and the sporulation rate in [%] was determined. None of the active component concentrations used inhibited the growth of the test strain. Sporulation was inhibited with increasing concentrations and was 95% suppressed at 450 μm.

TABLE 4

| | Concentration of patcouli alcohol [μm] | | |
|---|---|---|---|
| | 0 | 225 | 450 |
| Sporulation [%] | 100 | 95 | 5 |

### Example 3

Effect on Patchouli Oil on the Sporulation of Aspergillus niger on the Surface of an Acetate Jointing Compound

Commercially available, but preservative-free, one-component silicone jointing compounds curing at room temperature (acetate system, cured 2.2×2.2×0.3 cm pieces of film) were disinfected with 70% EtOH (ethanol) and placed for 24 h in active component solutions with various concentrations. The test specimens were then re-washed twice with EtOH, rinsed with distilled water (sterile) and dried for 24 h. The test specimens were weighed before and after this treatment and the quantity of active component in the test specimens was thus determined together with the concentration of the active component solution. The test specimens were then placed on wort agar plates and thinly coated with agar in which fungal spores had been incorporated (10⁵ CFU/ml Aspergillus niger, DSM 1988). The plates were incubated for 3 days at 25° C. Sporulation was visually evaluated from the test specimens and the sporulation rate in [%] was determined. None of the active-component concentrations tested inhibited the growth of the test strain. Sporulation was inhibited by increasing concentrations of eugenol and was completely suppressed at 9 μm/g jointing compound.

In a parallel test series with farnesol as active component, sporulation of the test strain was again inhibited, but to a far lesser extent compared with identical concentrations of patchouoli oil.

TABLE 5

| | Concentration of patchouli oil | | | | |
|---|---|---|---|---|---|
| | [μm/g jointing compound] | | | | |
| | 0 | 0.12 | 9 | 33 | 770 |
| Sporulation [%] | 100 | 100 | 0 | 0 | 0 |

TABLE 6
Control

| | Concentration of farnesol [μm/g jointing compound] | | | |
|---|---|---|---|---|
| | 0 | 0.4 | 1.1 | 17 |
| Sporulation [%] | 100 | 100 | 100 | 30 |

### Example 4

Effect of Patchouli Oil on the Sporulation of Aspergillus niger on a Filter Surface after 2 Applications

Filter papers (2×2 cm) were disinfected and treated twice with a 1 hour interval with 50 μl of active-component solutions (in ethanol) differing in concentration. The test specimens were then dried. The test specimens were then placed on wort agar plates and the surface of the wort agar plates was contaminated with 100 μl of a germ suspension (10³ CFU/ml) of Aspergillus niger (DSM 1988). The plates were incubated for 3 days at 25° C. Sporulation was visually evaluated and the sporulation rate in [%] was determined. None of the active-component concentrations tested inhibited the growth of the test strain. Sporulation was inhibited by increasing concentrations of eugenol and was 95% suppressed at 90 μm.

In a second, parallel test series with farnesol as the active component, sporulation of the test strain was again inhibited, but to a lesser extent compared with identical concentrations of patchouli oil.

TABLE 7

| | Concentration of patchouli oil [μm] | | | | | | | |
|---|---|---|---|---|---|---|---|---|
| | 0 | 0.5 | 5 | 25 | 35 | 50 | 70 | 90 |
| Sporulation [%] | 100 | 100 | 100 | 100 | 80 | 80 | 10 | 5 |
| | Concentration of farnesol [μm] | | | | | | | |
| | 0 | 0.5 | 5 | 25 | 35 | 50 | 70 | 90 |
| Sporulation [%] | 100 | 100 | 100 | 100 | 60 | 80 | 50 | 40 |

5. Liquid Detergent Containing Patchouli Oil

TABLE 8

| | Quantity |
|---|---|
| Raw material | in % by wt. |
| C₁₂-C₁₈ Fatty alcohol + 7 EO (Dehydol LT 7, Cognis) | 15 |
| C₁₂-C₁₄ Fatty alcohol C₁₂-C₁₈ fatty alcohol + 7 EO | 7 |
| (Dehydol LT 7, Cognis) + 2 EO sulfate, sodium salt | |
| (Texapon N 70, Cognis) | |
| C₈₋₁₈ Fatty acid, cut (coconut oil fatty acid, | 8 |
| Edenor K12-18, Cognis) | |
| Sodium citrate | 1.5 |
| Enzymes | + |
| Dye | + |
| Perfume | + |
| Patchouli oil (CAS 8014-09-3) | 0.4 |
| Water | to 100 |

6. Preportioned Liquid Detergent in Polyvinyl Alcohol Film Containing Patchouli Alcohol

TABLE 9

| | Quantity |
|---|---|
| Raw material | in % by wt. |
| C₁₂₋₁₄-Fatty alcohol + 5-EO + 4-PO (Marlox | 25 |
| MO 154, Sasol) | |
| Dodecyl benzenesulfonate Isopropylammonium salt | 24.5 |
| (LAS-MIPA, Sasol) | |
| C₈₋₁₈ Fatty acid, cut (coconut oil fatty acid, | 17.5 |
| Edenor K12-18, Cognis) | |
| Ethanol | 3.5 |
| Sodium citrate | 0.6 |
| Enzymes | 2.0 |
| Water | 6.0 |
| Patchouli alcohol | 0.6 |
| Dye | + |
| Perfume | + |
| Propylene glycol | to 100 |

The detergent is portioned in 50 ml doses.

7. Powder-Form Detergent Containing Patchouli Alcohol

TABLE 10

| | Quantity |
|---|---|
| Raw material | in % by wt. |
| C₁₀-C₁₃ Alkyl benzenesulfonate | 13.3 |
| C₁₂-C₁₈ Alkylsulfate | 5.5 |
| C₁₂-C₁₈ Alcohol + 7 EO | 5.3 |
| C₁₂-C₁₈ Alcohol + 4.5 EO | 0.6 |
| Soil Repellent | 0.7 |
| C₁₆-C₁₈ Fatty acid | 0.8 |
| (Edenor ST1 C₁₆-C₁₈, Cognis) | |
| Polyethylene glycol | 1.8 |
| Molecular weight = 4000 g/mol | |
| Phosphonate | 1.0 |
| Polyacrylatee | 2.8 |
| Carboxymethylcellulose | 0.9 |
| Polyvinylpyrrolidone | 0.5 |
| Zeolite (water-free active substance) | 32.1 |
| Sodium carbonat | 4.5 |
| Sodiumtricitrat | 3.6 |
| Citric acid | 3.7 |
| Sodium hydrogen carbonate | 4.9 |
| Sodium sulfate | 3.8 |
| Defoamer | + |
| Enzymes | + |
| Dye | + |
| Perfume | + |
| Patchouli alcohol | 0.4 |
| Water/salts | to 100 |

The detergent is packed in doses of 75 g.

Patchouli alcohol can also be incorporated as a constituent of the perfume. It is then present in the perfume oil in concentrations of 0.1 to 80% and is introduced into the wash liquor through the perfume oil present in the detergent formulation.

8. Mouth Wash

TABLE 11

| | % by weight |
|---|---|
| Ethanol (96%) | 65 |
| Polyoxyethylene sorbitan monolaurate (Tween ® | 2.0 |
| 20, Uniqema) | |
| Flavoring oil | 10.0 |
| Propylene glycol | 15.0 |
| Triethanolamine isostearate | 2.0 |
| Sodium saccharinate | 0.5 |
| Patchouli oil (CAS 8014-09-3) | 0.01 |
| Water | to 100 |

9. Toothpaste

TABLE 12

| | % by weight |
|---|---|
| Dicalcium phosphate | 47.5 |
| Glycerin 86% DAB | 30 |
| Toothpaste flavoring oil | 1.0 |
| Carboxymethyl cellulose, sodium salt | 1.2 |
| Sodium lauryl sulfate | 1.0 |
| Saccharin solution 1% | 0.5 |
| Patchouli oil (CAS 8014-09-3) | 0.02 |
| Water | to 100 |

10. Denture Cleaner, Powder-Form

TABLE 13

| | % by weight |
|---|---|
| Sodium perborate monohydrate | 25 |
| Sodium sesquicarbonate | 25 |
| Trisodium phosphate, water-free | 40 |
| Sodium lauryl sulfate | 0.2 |
| Silica | 0.5 |
| Flavors | 0.05 |
| Patchouli alcohol | 0.5 |
| Maltodextrin | 9.3 |

11. Denture Adhesive

TABLE 14

| | % by weight |
|---|---|
| Sodium alginate | 10 |
| Paraffin oil perliquidum | 90 |
| Patchouli alcohol | 0.01 |

12 to 15. Wallpaper Adhesives

TABLE 15

| Ingredients | Quantity | |
|---|---|---|
| Methylhydroxyethyl cellulose (300 mPas in 2% | 500 | g |
| aqueous solution, methoxyl content 26%) | | |
| PV Acetate redispersion powder | 350 | g |
| Kaolin | 60 | g |
| Cellulose powder | 50 | g |
| Addition product von 6 mol ethylene oxide onto 1 | 10 | g |
| mol nonyl phenol | | |
| Commercial preservative (based on isothiazoline | 8 | g |
| derivative) | | |
| Patchouli alcohol | 0.1 | g |

TABLE 16

| Ingredients | Quantity | |
|---|---|---|
| Methylhydroxyethyl cellulose (5000 mPas in | 680 | g |
| 2% aqueous solution, methoxyl content 19%) | | |
| Carboxylmethyl starch (DS 0.22) | 300 | g |
| Addition product von 4 mol ethylene oxide | 15 | g |
| onto 1 mol fatty alcohol | | |
| Commercial preservative (based on | 10 | g |
| isothiazoline derivative) | | |
| Patchouli alcohol | 0.1 | g |

TABLE 17

| Ingredients | Quantity | |
|---|---|---|
| Commercial polyvinyl acetate dispersions | 500 | g |
| (50% solids content) | | |
| Water | 200 | g |
| Methylhydroxyethyl cellulose (3000 mPas in | 20 | g |
| 2% aqueous solution) | | |
| Commercial preservative | 10 | g |
| Patchouli oil | 0.1 | g |

The mixtures obtained were made into pastes by stirring with water in a ratio of 1:20 (2) or 1:25 (3) or 1:1 (4) and used to hang commercially available wallpapers on walls.

We claim:

1. A method for reducing the adhesion of microorganisms to a surface comprising contacting the surface with at least one of patchouli oil, patchouli alcohol, or a derivative thereof.

2. The method of claim 1 wherein the surface is a textile, ceramic, metal, filter media, building material, building auxiliary, pelt, paper, skin, leather, or plastic.

3. The method of claim 1 wherein the surface is a surface that comes into contact with the human body.

4. The method of claim 3 wherein the surface that comes into contact with the human body is a surface of laundry, prostheses, or dentures.

5. The method of claim 1 wherein the microorganisms are bacteria or fungi.

6. The method of claim 5 wherein the microorganisms are fungi selected from molds, yeasts, and keratinophilic fungi.

7. The method of claim 6 wherein the fungi are molds of the genera Aspergillus, Penicillium, Cladosporium, or Mucor.

8. The method of claim 7 wherein the molds are Aspergillus aculeatus, Aspergillus albus, Aspergillus alliaceus, Aspergillus asperescens, Aspergillus awamori, Aspergillus candidus, Aspergillus carbonarius, Aspergillus carneus, Aspergillus chevalieri, Aspergillus chevalieri var. intermedius, Aspergillus clavatus, Aspergillus ficuum, Aspergillus flavipes, Aspergillus flavus, Aspergillus foetidus, Aspergillus fumigatus, Aspergillus giganteus, Aspergillus humicola, Aspergillus intermedius, Aspergillus japonicus, Aspergillus nidulans, Aspergillus niger, Aspergillus niveus, Aspergillus ochraceus, Aspergillus oryzae, Aspergillus ostianus, Aspergillus parasiticus, Aspergillus parasiticus var. globosus, Aspergillus penicillioides, Aspergillus phoenicis, Aspergillus rugulosus, Aspergillus sclerotiorum, Aspergillus sojae var. gymnosardae, Aspergillus sydowi, Aspergillus tamarii, Aspergillus terreus, Aspergillus terricola, Aspergillus toxicarius, Aspergillus unguis, Aspergillus ustus, Aspergillus versicolor, Aspergillus vitricola or Aspergillus wentii.

9. The method of claim 6 wherein the fungi are human pathogenic yeasts of the genus Candida.

10. The method of claim 6 wherein the fungi are keratinophilic fungi of the genera Malassezia, Trichophyton, Microsporum or Epidermophyton.

11. The method of claim 5 wherein the microorganisms are bacteria that are Propionibacterium acnes, Stapylococcus aureus, beta-haemolyzing Streptococci, S. pyogenes, Corynebacterium tenuis, Corynebacterium diphtheriae, Corynebacterium minutissimum, Micrococcus sedentarius, Bacillus anthracis, Neisseria meningitidis, N. gonorrhoeae, Pseudomonas aeruginosa, P. pseudomallei, Borrelia burgdorferi, Treponema pallidum, Mycobacterium tuberculosis, Mycobacterium spp., Escherichia coli, Streptococcus gordonii, Streptococcus mutans, Actinomyces naeslundii, Salmonella, Brachybacterium, Agrobacterium, Nitrosomonas, Aquabacterium, Hydrogenophaga, Stenotrophomonas, Xanthomonas, Neisseriaor Haemophilus.

12. The method of claim 1 wherein the microorganisms are human-, animal- or plant-pathogenic viruses or bacteriophages.

13. The method of claim 1 wherein the patchouli oil, patchouli alcohol, or derivative thereof is not biocidal, biostatic, or virus-inactivating in the final concentration used.

14. The method of claim 1 wherein the patchouli oil, patchouli alcohol, or derivative thereof is present on the surface in a concentration of 0.000001 % to 3% by weight.

15. A method for inhibiting the asexual propagation of fungi comprising contacting a material infested with the fungi with at least one of patchouli oil, patchouli alcohol, or a derivative thereof.

16. The method of claim 15 wherein the material infested with fungi is a textile, ceramic, metal, filter media, building material, building auxiliary, pelt, paper, skin, leather, or plastic.

17. The method of claim 15 wherein the patchouli oil, patchouli alcohol, or derivative thereof is not biocidal, biostatic, or virus-inactivating in the final concentration used.

18. The method of claim 15 wherein the patchouli oil, patchouli alcohol, or derivative thereof is present in the material infested with the fungi in a concentration of 0.000001 % to 3% by weight.

19. A method for treating a microbial infection comprising topically applying at least one of patchouli oil, patchouli alcohol, or a derivative thereof to a patient suffering from such an infection.

20. The method of claim 19 wherein the microbial infection is a fungal infection.

21. The method of claim 20 wherein fungal infection is caused by keratinophilic fungi.
